(19) United States
(12) Patent Application Publication
(10) Pub. No.: US 2025/0017915 A1
(43) Pub. Date: Jan. 16, 2025

(54) METHOD OF TREATING CANCER ASSOCIATED WITH RAS MUTATION

(71) Applicants: The Board of Trustees of the University of Illinois, Urbana, IL (US); The United States Government as Represented by The Department of Veterans Affairs, Washington, DC (US)

(72) Inventors: Gregory R. Thatcher, Tucson, AZ (US); Ajay Rana, Chicago, IL (US); Rui Xiong, Chicago, IL (US); Daniel Principe, Vernon Hills, IL (US); Yangfeng Li, Chicago, IL (US)

(21) Appl. No.: 18/710,037

(22) PCT Filed: Nov. 15, 2022
(86) PCT No.: PCT/US2022/079848

§ 371 Date: May 14, 2024

Related U.S. Application Data
(60) Provisional application No. 63/279,236, filed on Nov. 15, 2021.

(51) Int. Cl. A61K 31/444; A61K 39/395; A61P 35/00

(52) U.S. Cl. CPC A61K 31/444; A61K 39/3955; A61P 35/00

(57) ABSTRACT

In accordance with the purpose(s) of the present disclosure, described herein are methods for treating cancer associated with a Ras mutation in a subject in need of treatment comprising administering to the subject an effective amount of a compound of formula I as described herein or a pharmaceutically acceptable salt thereof. The compounds described herein have improved efficacy compared to the benchmark BET inhibitor JQ-1 and comparable efficacy to high dose JQ-1 with respect to the treatment of PDAC. In one aspect, the compounds are administered in combination with an effective amount of an immune checkpoint inhibitor such as, for example, PD-1/PD-L 1 inhibitor, a CTLA-4 inhibitor, or a combination thereof.

### CROSS-REFERENCE TO RELATED APPLICATIONS

This application claims the benefit of and priority to co-pending U.S. Provisional Patent Application No. 63/279,236, filed on Nov. 15, 2021, the contents of which are incorporated by reference herein in their entireties.

### STATEMENT REGARDING FEDERALLY SPONSORED RESEARCH OR DEVELOPMENT

This invention was made with government support under grant nos. I01BX004903 and IK6 BX004855 awarded by the United States Department of Veterans Affairs. The government has certain rights in this invention.

### BACKGROUND

Mutation of Ras genes has been frequently observed in cancer patients. Mutant RAS is a driver of tumor initiation and maintenance. There are three human RAS genes: Kirsten rat sarcoma viral oncogene homolog (KRAS), neuroblastoma RAS viral (v-ras) oncogene homolog (NRAS), and Harvey rat sarcoma viral oncogene homolog (HRAS)] encode four RAS proteins, with two KRAS isoforms that arise from alternative RNA splicing (KRAS4A and KRAS4B). RAS proteins (KRAS4A, KRAS4B, NRAS and HRAS) function as GDP-GTP-regulated binary on-off switches, which regulate cytoplasmic signaling networks that control diverse normal cellular processes.

One example of a cancer associated with a Ras mutation is pancreatic ductal adenocarcinoma (PDAC). There are currently no curative therapies for patients with advanced PDAC. While broad-spectrum chemotherapy can modestly extend survival, nearly all patients will eventually progress on treatment, and overall outcomes remain poor (34). Additionally, PDAC has shown poor clinical responses to newly emerging treatments, including immunotherapy, in clinical trials (35, 36). Thus, there is a need for new approaches and treatments for PDAC and other cancers associated with a Ras mutation.

### SUMMARY

In accordance with the purpose(s) of the present disclosure, described herein are methods for treating cancer associated with a Ras mutation in a subject in need of treatment comprising administering to the subject an effective amount of a compound of formula I or a pharmaceutically acceptable salt thereof

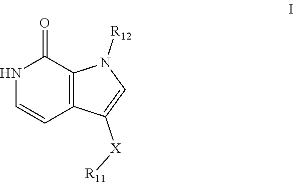

wherein
R₁₂ is a C₁-C₃ alkyl, a C₁-C₃ haloalkyl, propylenyl, —CH₂(CO)CH═CH₂, oxiran-2-ylmethyl, or CH₂(CO)CH₂Cl;
R₁₁ is a nitrogen-containing bicyclic or tricyclic heteroaryl, an aryl, or a biaryl, each of which is optionally substituted with 1, 2, or 3 substituents independently selected from —N(R^{a})S(O)₂R^{b}, —S(O)₂NR^{a}R^{b}, —C(O)NR^{a}R^{b}—N(R^{a})C(O)R^{b} —NR^{a}R^{b}, —(C₁-C₆ alkylenyl)R^{c}, —(C₁-C₃ cycloalkylenyl)R^{c}, aryl, heteroaryl, —(C₁-C₆ alkylenyl)R^{c}R^{c′}, —H, halogen, —CN, propylenyl, C₁-C₃ alkyl, C₁-C₃ haloalkyl, —OR₇₀, —NR₇₀R₇₀, —C(O)OR₇₀, —C(O)NR₇₀R₇₀, —S(O)₂R₇₀, —S(O)₂NR₇₀R₇₀, —CH₂(CO)CH═CH₂, oxiran-2-ylmethyl, CH₂(CO)CH₂Cl, and R₇₀;
X is optionally present, and when present, is selected from —O—, —C(O)—, —N(R₇₇)—, and —CH(R₇₀)—,
R₇₇ is selected from the group consisting of: —H, a halogen, —CN, C₁-C₃ haloalkyl, —OR₇₀, —NR₇₀R₇₀, —C(O)OR₇₀, —C(O)NR₇₀R₇₀, —S(O)₂R₇₀, —S(O)₂NR₇₀R₇₀, and R₇₀;
R₇₀, at each occurrence, are each independently selected from C₁-C₆ alkyl, C₂-C₆ alkenyl, C₂-C₆ alkynyl, halogen, C₁-C₆ haloalkyl, —CN, NO₂, —OR^{e}, —S(O)₂NR^{e}R^{f}, —C(O)R^{e}, —C(O)NR^{e}R^{f}, —NR^{e}R^{f}, —N(R^{e})C(O)R^{f}, —(C₁-C₆ alkylenyl)-OR^{e}, —(C₁-C₆ alkylenyl)-C(O)NR^{e}R^{f}, a —(C₁-C₆alkylenyl)-NR^{e}R^{f}, and —(C₁-C₆ alkylenyl)-N(R^{e})C(O)R^{f};
R^{a} and R^{b}, at each occurrence, are independently selected from H, C₁-C₆ alkenyl, C₁-C₆ alkynyl, C₁-C₆ haloalkyl, R^{c}, and C₁-C₆ alkyl, wherein the C₁-C₆ alkyl is optionally substituted with one substituent selected
   from —OR^{e}, —NR^{e}R^{f}, —C(O)OR^{e}, —C(O)NR^{e}R^{f}, —S(O)₂R^{e}, —S(O)₂NR^{e}R^{f}, and R^{c};
R^{c} and R^{c′}, at each occurrence, are independently selected from aryl, heteroaryl, heterocycle, cycloalkyl, and cycloalkenyl, wherein each R^{c} group is optionally substituted with 1, 2, 3, 4, or 5 R^{d} groups;
R^{d}, at each occurrence, are independently selected from halogen, C₁-C₆ alkyl, C₂-C₆ alkenyl, C₂-C₆ alkynyl, C₁-C₆ haloalkyl, —CN, —NO₂, —OR^{e}, —S(O)₂NR^{e}R^{f}, —C(O)R^{e}, —C(O)NR^{e}R^{f}, —NR^{e}R^{f}, —N(R^{e})C(O)R^{f}, —(C₁-C₆ alkylenyl)-OR^{e}, —(C₁-C₆ alkylenyl)-C(O)NR^{e}R^{f}, —(C₁-C₆ alkylenyl)-NR^{e}R^{f}, and —(C₁-C₆ alkylenyl)-N(R^{e})C(O)R^{f}; and
R^{e} and R^{f}, at each occurrence, are independently selected from H, C₁-C₆ alkyl, C₁-C₆ cycloalkyl, aryl, heteroaryl and C₁-C₆ haloalkyl.

The compounds described herein have improved efficacy compared to the benchmark BET inhibitor JQ-1 and comparable efficacy to high dose JQ-1. In one aspect, the compounds are administered in combination with an effective amount of an immune checkpoint inhibitor such as, for example, PD-1/PD-L1 inhibitor, a CTLA-4 inhibitor, or a combination thereof.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1G show that BRD4 and EP300/CBP are ubiquitously expressed in PDAC. (A, B) Excisional biopsies from 14 PDAC patients, 9 with matched adjacent non-malignant tissue, were sectioned and stained for either BRD4 or EP300/CBP. The number of positive nuclei per 40× field was quantified by three blinded investigators and divided by the total number of nuclei in each field. These values were averaged and displayed as an individual value plot. (C,D) Pancreatic tumor tissues (PDAC) and adjacent non-malignant (N) The Badea et al. and Pei et al. cohorts of PDAC patients were evaluated for mRNA expression of BRD4 or EP300 using the Oncomine platform. All mRNA expression values are plotted in log scale. (E) Human PDAC cell lines Panc1, ASPC1, MiaPaCa2, Capan1, and the murine PDAC cell line KPC-105 were evaluated for expression of BRD4 and EP300/CBP by western blot. (F) Panc1 cells were stained for BRD4 and EP300/CBP by immunocytochemistry. (G) Pancreas tissue from either non-genic wild type (WT), the Pdx1-Cre×LSL-Kras^{G12D} (KC) model of PanIN disease, the Pdx1-Cre×LSL-Kras^{G12D}×LSL-TP53^{R172H+/-}(KPC) model of advanced PDAC, the Pdx1-Cre×LSL-Kras^{G12D}×LSL-TP53^{R172H+/+}(KPPC) model of extremely aggressive PDAC, or subcutaneous tumor tissue from the G-68 cell derived xenograft (CDX) model were collected and stained for either BRD4 or EP300/CBP. (*p<0.05)

FIGS. 2A-2G show that XP-524 is a potent, multi-specificity BET inhibitor that engages BRD4 and EP300/CBP. (A) Chemical structure of XP-524, designed to function as a multi-specificity BET inhibitor with activity against EP300/CBP. (B,C) TR-FRET assay using either the first (BD1) or second (BD2) bromodomain of BRD4 and increasing concentrations of either the first generation BET inhibitor JQ-1 or XP-524. (D) BROMOscan assay using EP300 or its structural analog CBP and increasing concentrations of XP-524. (E) Co-crystal structure of XP-524:BRD4 showing extensive interactions between XP-524 and the acetylated lysine pocket of the BD1 bromodomain (PDB: 6P05). (F) Co-crystal structure of XP-524 with EP300 analog CBP (XP-524:CBP) showing extensive interactions between XP-524 and the acetylated lysine pocket of the CBP bromodomain, as well as bidentate hydrogen-bonding with Asn-1168 and insertion of the sulfate moiety into the ZA loop forming a hydrogen bond with the backbone of Asp-1116 (PDB: 7JUO). (G) Overlay of the XP-524-CBP complex and the XP-524-BRD4 complex showing a similar binding mode.

FIGS. 3A-3P show that inhibition of EP300/CBP potentiates BETi-mediated silencing of oncogenic KRAS signaling. (A) An equal number of Panc1 cells were seeded in 24 well plates and treated with either a DMSO vehicle, 1 μM JQ-1, JQ-1 and 1 μM of SGC-CBP30, 1 μM XP-524, or XP-524 and SGC-CBP30. Cell growth was evaluated every four hours until the control group reached 100% confluence (N=4/group). (B) Panc1 cells were incubated with increasing concentrations of either JQ-1 or XP-524, each with or without a fixed 1 μM dose of SGC-CBP30. After 48 hours cell viability was evaluated by MTT assay. (C) Panc1 cells were incubated with either a DMSO vehicle, 1 μM JQ-1, JQ-1 and 1 μM SGC-CBP30, or 1 μM XP-524 and subjected to RNA sequencing. (D) Focused heatmap showing select, significantly altered genes in the cell cycle pathway using a false discovery rate (FDR) adjusted p value of <0.05. (E) Focused heatmap showing select, significantly altered genes in the KRAS signaling pathway using a FDR adjusted p value of <0.05. (F-H) Panc1, MiaPaCa2 (MP2), and ASPC1 cells were treated with 1 μM XP-524 and KRAS mRNA evaluated by qPCR. Values are presented as fold change compared to DMSO control (C) treated samples. (1) Panc1 cells were again treated with 1 μM JQ-1, JQ-1 and 1 μM SGC-CBP30, or 1 μM XP-524 and the interaction between BRD4 and EP300 evaluated by immunoprecipitation (IP) followed by western blot (IB). (J,K) Panc1 and KPC-105 cells were treated as described and evaluated by western blot for pRB, H3K27 acetylation, as well as KRAS expression and downstream activation of the MEK/ERK pathway. (L) MiaPaCa2 (MP2) and ASPC1 cells and KRAS expression and downstream MEK/ERK activation evaluated by western blot. (I-K) Excisional biopsies from three PDAC patients undergoing survival resection were cored, sectioned at 250 μm interval, and cultured ex vivo either in a control PBS vehicle or 5 μM XP-524. After 72 hours, slice cultures were formalin fixed, paraffin embedded, and stained with H&E or by immunohistochemistry for pERK or CK19 and PCNA. (*p<0.05).

FIGS. 4A-4G show that XP-524 reduces mutant KRAS-induced PanIN formation in vivo. (A) Ptf1a-Cre×LSL-Kras^{G12D+/-} (KPC) mice were generated as a model of early pancreatic intraepithelial neoplasms (PanINs). Starting at 8 weeks of age, mice were administered daily intraperitoneal injections of either PBS vehicle or 5 mg/kg XP-524 and sacrificed at a fixed endpoint of 6 months. (B) At the study endpoint, the pancreas gland was weighted, normalized to each animal's body weight, and results displayed as individual value plots. (C,D) Pancreas tissues were stained with H&E, Masson's Trichrome, or via immunohistochemistry for CK19 and pancreatic amylase, quantified as described, and results displayed as individual value plots. (E,F) Tissues were also stained by immunohistochemistry for mutant RAS^{G12D}, pERK, or CK19 and PCNA. Results were quantified as described and displayed as individual value plots. (G) Pancreas tissues were lysed at the study endpoint and evaluated by western blot for KRAS expression and downstream activation of the MEK/ERK pathway. (*p<0.05).

FIGS. 5A-5H show that XP-524 extends survival and reduces pathologic KRAS activation in murine PDAC. (A) Pdx1-Cre×LSL-Kras^{G12D}×LSL-TP53^{R172H+/-} (KPC) mice were generated as a model of advanced PDAC. Starting at 15 weeks of age, mice were administered daily intraperitoneal injections of either PBS vehicle or 5 mg/kg XP-524. Pancreas tissues were collected when the animals were moribund. (B) Kaplan-Meier curve indicating survival for mice across both groups in days post enrollment (N=7/group). (C,D) At the study endpoint, gross changes in pancreas gland structure were evaluated, including gland weight, which was also normalized to each animal's body weight, and results displayed as individual value plots. Tissues were stained with H&E or via immunohistochemistry for pERK, E-Cadherin and PCNA, or Cleaved Caspase 3, quantified as described and results displayed as individual value plots. (E) Pdx1-Cre×LSL-Kras^{G12D}×LSL-TP53^{R172H+/+} (KPPC) mice were generated as a model of extremely aggressive PDAC. Mice were enrolled either at 2 weeks of age, or when developing a 0.5 cm³ tumor. (F) Kaplan-Meier curve indicating survival for mice across both groups in days post enrollment (N=6/group). (G,H) At the study endpoint, gross changes in pancreas gland structure were evaluated, including gland weight, which was also normalized to each animal's body weight, and results displayed as individual value plots. Tissues also were stained with H&E or via immunohistochemistry for pERK, E-Cadherin and PCNA, or Cleaved Caspase 3, quantified as described and results displayed as individual value plots. (*p<0.05)

FIGS. 6A-6L show that XP-524 enhances T-cell recruitment but fails to promote a functional anti-tumor immune response. (A,B) Pdx1-Cre×LSL-Kras^{G12D}×LSL-TP53^{R172H+/-} (KPC) mice were generated as a model of advanced PDAC. Starting at 15 weeks of age, mice were administered daily intraperitoneal injections of either PBS vehicle or 5 mg/kg XP-524. Pancreas tissues were collected when the animals were moribund and stained with either H&E or via immunohistochemistry for E-Cadherin and the T-cell surrogate CD3, or for CD3 and T-cell exhaustion marker PD-1. Tissue sections were quantified as described and displayed as an individual value plot. (C) KPC were enrolled as described and treated with either a PBS vehicle or 5 mg/kg XP-524. After two months on treatment, tumor tissues were collected and analyzed by flow cytometry for tumor infiltrating CD4+ and CD8+ T-cells, respectively (N=4/group). (D) Tumor infiltrating cells were gated based on the CD8 staining shown previously, and the total number of cells positive for IFNγ displayed as an individual value plot. (E) The total number of cells positive for either CD4 or CD8 and T-cell exhaustion marker PD-1 from the tumors of PBS and XP-524 treated mice. (F) Tumor infiltrating T-cells were gated based on CD4 staining, and analyzed for CD25 and FoxP3, and the total number of CD4+CD25+FoxP3 regulatory T-cells displayed by as individual value plot. (G, H) Panc1 cells were treated with either a DMSO vehicle or 1 μM XP-524, analyzed by RNA sequencing, and subjected to gene set enrichment analysis revealing a significant enrichment for the antigen processing and presentation pathway. (I) Tissues from KPC mice treated with either PBS or XP-524 were stained via immunohistochemistry for MHC Class 1 and representative images displayed. (J,K) RNA sequencing and gene set enrichment analysis for Panc1 treated as described, revealing a significant downregulation of the TGFβ signaling pathway. (L) Homogenized tumor tissue or serum from KPC mice treated with either PBS or XP-524 were analyzed for levels of TGFβ1 by ELISA. (*p<0.05)

FIGS. 7A-7L show that XP-524 cooperates with PD-1 inhibition to further extend survival in KPC mice. (A) Pdx1-Cre×LSL-Kras^{G12D}×LSL-TP53^{R172H+/-} (KPC) mice were generated as a model of advanced PDAC. Starting at 15 weeks of age, mice were administered either twice weekly intraperitoneal injections of 200 μg of anti-PD-1 (N=7), or daily injections of 5 mg/kg XP-524 with twice weekly injections of anti-PD-1 (N=11). The study was concluded when animals showed signs of disease associated morbidity. (B) Kaplan-Meier curve indicating survival for mice across both groups in days post enrollment. (C-E) At the study endpoint, pancreas tissues were collected and stained with either H&E or via immunohistochemistry for pan-leukocyte marker CD45 or for E-Cadherin and the T-cell surrogate CD3. Tissues were quantified as described and counts displayed as individual value plots. (F) Tumor tissues were also stained for the cytotoxic T-cell marker CD8, CK19 and GranzymeB, or the apoptosis surrogate Cleaved Caspase 3. (G) KPC mice were enrolled as described and treated with either anti-PD-1 or XP-524 and anti-PD-1. After two months on treatment, tumor tissues were collected and analyzed by flow cytometry for tumor infiltrating CD4+ and CD8+ T-cells, respectively (N=4/group). (H) Tumor infiltrating cells were gated based on the CD8 staining shown previously, and the total number of cells positive for IFNγ displayed as an individual value plot. (I) The total number of cells positive for either CD4 or CD8 and T-cell exhaustion marker PD-1 from the tumors of anti-PD-1 and XP-524+ anti-PD-1 treated mice. (J) Tumor infiltrating T-cells were gated based on CD4 staining, and analyzed for CD25 and FoxP3, and the total number of CD4+CD25+FoxP3 regulatory T-cells displayed by as individual value plot. (K) Spleens and tumor infiltrating cells were gated based on the CD8 and CD4 staining shown above, and CD8+ events isolated and analyzed for expression of cytotoxic T-cell activation marker Perforin. The total numbers of cells positive for both CD8 and Perforin are displayed as individual value plots. (L) Tumor infiltrating CD8+ cells were gated as previously, and analyzed for the simultaneous expression of the aforementioned T-cell activation markers including Perforin and IFNγ. The number of CD8+Perforin+IFNγ+ cells from each group is displayed as an individual value plot. (*p<0.05).

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “an excipient” include, but are not limited to, mixtures or combinations of two or more such excipients, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range. Thus, for example, if a component is in an amount of about 1%, 2%, 3%, 4%, or 5%, where any value can be a lower and upper endpoint of a range, then any range is contemplated between 1% and 5% (e.g., 1% to 3%, 2% to 4%, etc.).

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, “IC₅₀,” is intended to refer to the concentration of a substance (e.g., a compound or a drug) that is required for 50% inhibition of a biological process, or component of a process. For example, IC₅₀ refers to the half maximal (50%) inhibitory concentration (IC) of a substance as determined in a suitable assay.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

The position of a substituent can be defined relative to the positions of other substituents in an aromatic ring. For example, as shown below in relationship to the “R” group, a second substituent can be “ortho,” “para,” or “meta” to the R group, meaning that the second substituent is bonded to a carbon labeled ortho, para, or meta as indicated below. Combinations of ortho, para, and meta substituents relative to a given group or substituent are also envisioned and should be considered to be disclosed.

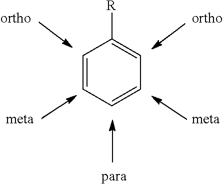

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “alkanediyl” as used herein, refers to a divalent saturated aliphatic group, with one or two saturated carbon atom(s) as the point(s) of attachment, a linear or branched, cyclo, cyclic or acyclic structure, no carbon-carbon double or triple bonds, and no atoms other than carbon and hydrogen. The groups, —CH₂— (methylene), —CH₂CH₂—, —CH₂C(CH₃)₂CH₂—, and —CH₂CH₂CH₂— are non-limiting examples of alkanediyl groups.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cyclooctynyl, cyclononynyl, and the like. The term “heterocycloalkynyl” is a type of cycloalkenyl group as defined above and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the π clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl to two aryl groups that are bound together via a fused ring structure, as in naphthalene, or are attached via one or more carbon-carbon bonds, as in biphenyl. Fused aryl groups including, but not limited to, indene and naphthalene groups are also contemplated.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) and —N(-alkyl)₂, where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein.

The terms “halo,” “halogen” or “halide,” as used herein can be used interchangeably and refer to F, Cl, Br, or I.

The terms “pseudohalide,” “pseudohalogen” or “pseudohalo,” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl,” “heteroaryl,” “bicyclic heterocycle,” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxyl” or “hydroxy” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃.

The term “nitro” as used herein is represented by the formula —NO₂.

The term “nitrile” or “cyano” as used herein is represented by the formula —CN.

The term “silyl” as used herein is represented by the formula —SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A'S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A'S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” . . . “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or l meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, sulfur, fluorine and chlorine, such as ²H, ³H, ¹³C, ¹⁴C ¹⁵N, ¹⁸O, ¹⁷O ³⁵S, ¹⁸F, and ³⁶Cl, respectively. Compounds further comprise prodrugs thereof and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

It is also appreciated that certain compounds described herein can be present as an equilibrium of tautomers. For example, ketones with an α-hydrogen can exist in an equilibrium of the keto form and the enol form.

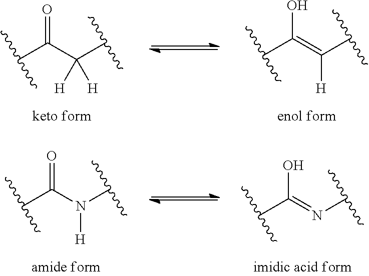 [SMILES: *CC(*)=O ; */C=C(/*)O ; *NC(*)=O ; */N=C(/*)O]

Likewise, amides with an N-hydrogen can exist in an equilibrium of the amide form and the imidic acid form. Unless stated to the contrary, the invention includes all such possible tautomers.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

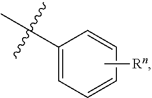

which is understood to be equivalent to a formula:

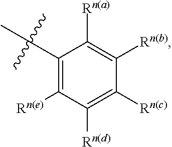

wherein n is typically an integer. That is, Rⁿ is understood to represent five independent substituents, R^{n(a)}, R^{n(b)}, R^{n(c)}, R^{n(d)}, and R^{n(e)}. By “independent substituents,” it is meant that each R substituent can be independently defined. For example, if in one instance R^{n(a)} is halogen, then R^{n(b)} is not necessarily halogen in that instance.

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as a hematological malignancy, breast cancer, and/or another solid malignancy. The effect can be therapeutic in terms of a partial or complete cure of a disease, condition, symptom or adverse effect attributed to the disease, disorder, or condition. The term “treatment” as used herein can include any treatment of a hematological malignancy, breast cancer, and/or another solid tumor in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “therapeutic” can refer to treating, healing, and/or ameliorating a disease, disorder, condition, or side effect, or to decreasing in the rate of advancement of a disease, disorder, condition, or side effect.

As used herein, “effective amount” can refer to the amount of a disclosed compound or pharmaceutical composition provided herein that is sufficient to effect beneficial or desired biological, emotional, medical, or clinical response of a cell, tissue, system, animal, or human. An effective amount can be administered in one or more administrations, applications, or dosages. The term can also include within its scope amounts effective to enhance or restore to substantially normal physiological function.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound and/or pharmaceutical composition, for example, can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

The term “pharmaceutically acceptable” describes a material that is not biologically or otherwise undesirable, i.e., without causing an unacceptable level of undesirable biological effects or interacting in a deleterious manner.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

The term “pharmaceutically acceptable prodrug” or “prodrug” represents those prodrugs of the compounds of the present disclosure which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the present disclosure can be rapidly transformed in vivo to a parent compound having a structure of a disclosed compound, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

The term “pharmaceutically acceptable vehicle” refers to a diluent, adjuvant, excipient or carrier with which a compound of the disclosure is administered. The terms “effective amount” or “pharmaceutically effective amount” refer to a nontoxic but sufficient amount of the agent to provide the desired biological result. That result can be reduction and/or alleviation of the signs, symptoms, or causes of a disease, or any other desired alteration of a biological system. An appropriate “effective” amount in any individual case can be determined by one of ordinary skill in the art using routine experimentation.

“Pharmaceutically acceptable carriers” for therapeutic use are well known in the pharmaceutical art, and are described, for example, in Remington's Pharmaceutical Sciences, 18th Edition (Easton, Pennsylvania: Mack Publishing Company, 1990). For example, sterile saline and phosphate-buffered saline at physiological pH can be used. Preservatives, stabilizers, dyes and even flavoring agents can be provided in the pharmaceutical composition. For example, sodium benzoate, sorbic acid and esters of p-hydroxybenzoic acid can be added as preservatives. Id. at 1449. In addition, antioxidants and suspending agents can be used. Id.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

The term “modulating” or “modulate” refers to the treating, prevention, suppression, enhancement or induction of a function, condition or disorder. For example, it is believed that the compounds of the present disclosure can modulate PDAC by inhibiting the BET protein BRD4 and the histone acetyltransferase EP300/CBP, both of which are ubiquitously expressed in PDAC tissues and cooperate to enhance tumorigenesis.

The term “contacting” as used herein refers to bringing a disclosed compound or pharmaceutical composition in proximity to a cell, a target protein, or other biological entity together in such a manner that the disclosed compound or pharmaceutical composition can affect the activity of the a cell, target protein, or other biological entity, either directly; i.e., by interacting with the cell, target protein, or other biological entity itself, or indirectly; i.e., by interacting with another molecule, co-factor, factor, or protein on which the activity of the cell, target protein, or other biological entity itself is dependent.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e. one atmosphere).

Methods of Treating Cancer Associated with a Ras Mutation

Described herein are methods for treating cancer associated with a Ras mutation in a subject in need of treatment. Mutant RAS is a driver of tumor initiation and maintenance. There are three human RAS genes: Kirsten rat sarcoma viral oncogene homolog (KRAS), neuroblastoma RAS viral (v-ras) oncogene homolog (NRAS), and Harvey rat sarcoma viral oncogene homolog (HRAS)] encode four RAS proteins, with two KRAS isoforms that arise from alternative RNA splicing (KRAS4A and KRAS4B). RAS proteins (KRAS4A, KRAS4B, NRAS and HRAS) function as GDP-GTP-regulated binary on-off switches, which regulate cytoplasmic signaling networks that control diverse normal cellular processes.

In one aspect, the Ras gene is mutated at least 15%, at least 20%, or at least 30%. Cancers associated with a Ras gene mutation include lung cancer, gastrointestinal cancer, thoracic cancer, pancreatic cancer, colon cancer, or haematologic cancer. In another aspect, the cancer associated with a Ras mutation is small intestine adenocarcinoma, rectal adenocarcinoma, cholangiocarcinoma, gallbladder carcinoma, neuroblastoma, pancreatic ductal adenocarcinoma (PDAC), or melanoma.

In pancreatic ductal adenocarcinoma (PDAC), bromodomain and extra-terminal motif (BET) proteins contribute to disease pathogenesis predominantly through the transcriptional activation of several oncogenes, including c-MYC and FOSL1. Additionally, the EP300 bromodomain is essential for oncogenic cMYC expression and cell proliferation.

With that said, described herein are compounds and formulations thereof that can modulate the activity of the bromodomain and extraterminal (BET) family of bromodomains and bromodomain proteins for the treatment of PDAC. In one aspect, the compounds described herein inhibits EP300 and CBP (a structural analog of EP300) proteins with bromodomain 4 (BRD4). In another aspect, the compounds described herein suppress the acetylation of H3K27, a known action of EP300.

In another aspect, the compounds described herein can suppress oncogenic KRAS signaling. KRAS mutations are observed in over 90% of PDAC patients, resulting in permanent activation of the KRAS protein, which drives several cancer-associated cellular processes, including proliferation, transformation, invasion, and survival (41-44). Oncogenic KRAS mutations have long been considered an early event in PDAC etiology, and sustained KRAS activity is required for both the initiation and maintenance of the neoplastic phenotype (45). Despite the well-established role of oncogenic KRAS in PDAC, KRAS has proven largely undruggable in the clinic (46). While most therapies directed against KRAS have sought to directly interfere with the KRAS protein directly (46), the compounds described herein inhibit BRD4 and EP300, which impedes KRAS transcription and restrains its downstream signaling.

The compounds described herein have improved efficacy compared to the benchmark BET inhibitor JQ-1 and comparable efficacy to high dose JQ-1. In one aspect, the compounds are administered in combination with an effective amount of an immune checkpoint inhibitor such as, for example, PD-1/PD-L1 inhibitor, a CTLA-4 inhibitor, or a combination thereof.

### Pyridinone-Based Compounds

In one aspect, the compounds described herein have the formula I or a pharmaceutically acceptable salt thereof

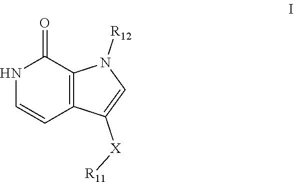

wherein
R₁₂ is a C₁-C₃ alkyl, a C₁-C₃ haloalkyl, propylenyl, —CH₂(CO)CH═CH₂, oxiran-2-ylmethyl, or CH₂(CO)CH₂Cl;
R₁₁ is a nitrogen-containing bicyclic or tricyclic heteroaryl, an aryl, or a biaryl, each of which is optionally substituted with 1, 2, or 3 substituents independently selected
   from —N(R^{a})S(O)₂R^{b}, —S(O)₂NR^{a}R^{b}, —C(O)NR^{a}R^{b}—N(R^{a})C(O)R^{b} —NR^{a}R^{b}, —(C₁-C₆ alkylenyl)R^{c}, —(C₁-C₃ cycloalkylenyl)R^{c}, aryl, heteroaryl, —(C₁-C₆ alkylenyl)R^{c}R^{c′}, —H, halogen, —CN, propylenyl, C₁-C₃ alkyl, C₁-C₃ haloalkyl, —OR₇₀, —NR₇₀R₇₀, —C(O)OR₇₀, —C(O)NR₇₀R₇₀, —S(O)₂R₇₀, —S(O)₂NR₇₀R₇₀, —CH₂(CO )CH═CH₂, oxiran-2-ylmethyl, CH₂(CO)CH₂Cl, and R₇₀; X is optionally present, and when present, is selected from —O—, —C(O)—, —N(R₇₇)—, and —CH(R₇₀)—,
R₇₇ is selected from the group consisting of: —H, a halogen, —CN, C₁-C₃ haloalkyl, —OR₇₀, —NR₇₀R₇₀, —C(O)OR₇₀, —C(O)NR₇₀R₇₀, —S(O)₂R₇₀, —S(O)₂NR₇₀R₇₀, and R₇₀;
R₇₀, at each occurrence, are each independently selected from C₁-C₆ alkyl, C₂-C₆alkenyl, C₂-C₆ alkynyl, halogen, C₁-C₆ haloalkyl, —CN, NO₂, —OR^{e}, —S(O)₂NR^{e}R^{f}, —C(O)R^{e}, —C(O)NR^{e}R^{f}, —NR^{e}R^{f}, —N(R^{e})C(O)R^{f}, —(C₁-C₆ alkylenyl)-OR^{e}, —(C₁-C₆ alkylenyl)-C(O)NR^{e}R^{f}, a —(C₁-C₆alkylenyl)-NR^{e}R^{f}, and —(C₁-C₆ alkylenyl)-N(R^{e})C(O)R^{f};
R^{a} and R^{b}, at each occurrence, are independently selected from H, C₁-C₆ alkenyl, C₁-C₆ alkynyl, C₁-C₆ haloalkyl, R^{c}, and C₁-C₆ alkyl, wherein the C₁-C₆ alkyl is optionally substituted with one substituent selected
   from —OR^{e}, —NR^{e}R^{f}, —C(O)OR^{e}, —C(O)NR^{e}R^{f}, —S(O)₂R^{e}, —S(O)₂NR^{e}R^{f}, and R^{c};
R^{c} and R^{c′}, at each occurrence, are independently selected from aryl, heteroaryl, heterocycle, cycloalkyl, and cycloalkenyl, wherein each R^{c} group is optionally substituted with 1, 2, 3, 4, or 5 R^{d} groups;
R^{d}, at each occurrence, are independently selected from halogen, C₁-C₆ alkyl, C₂-C₆ alkenyl, C₂-C₆ alkynyl, C₁-C₆ haloalkyl, —CN, —NO₂, —OR^{e}, —S(O)₂NR^{e}R^{f}, —C(O)R^{e}, —C(O)NR^{e}R^{f}, —NR^{e}R^{f}, —N(R^{e})C(O)R^{f}, —(C₁-C₆ alkylenyl)-OR^{e}, —(C₁-C₆ alkylenyl)-C(O)NR^{e}R^{f}, —(C₁-C₆ alkylenyl)-NR^{e}R^{f}, and —(C₁-C₆ alkylenyl)-N(R^{e})C(O)R^{f}; and
R^{e} and R^{f}, at each occurrence, are independently selected from H, C₁-C₆ alkyl, C₁-C₆ cycloalkyl, aryl, heteroaryl and C₁-C₆ haloalkyl.

In one aspect, the compound is Formula II

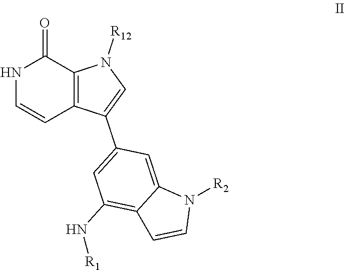

wherein R₁ is

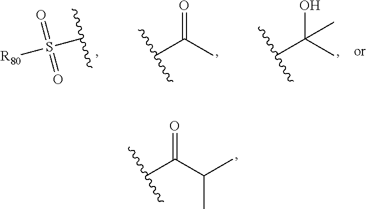

wherein R₂ is

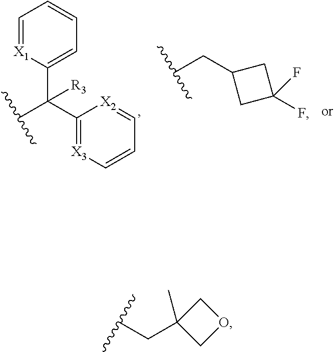

wherein R₃ is H or C₁-C₆ alkyl,
wherein R₈₀ is C₁-C₃ alkyl,
wherein X₁, X₂, and X₃, are each independently selected from the group consisting of: CH or N.

In one aspect, R₁₂ in formula II is a C₁-C₃alkyl such as, for example, methyl. In another aspect, R₂ in formula II is

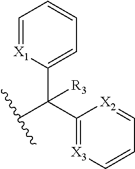

wherein X₁ is N, X₂ is N, and X₃ is CH. In another aspect, R₃ in formula II is a C₁-C₃ alkyl such as, for example, methyl. In another aspect, R₁ in formula II is

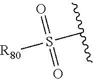

wherein R₈₀ in formula II is a C₁-C₃ alkyl such as, for example, ethyl.

In another aspect, the compound is Formula III

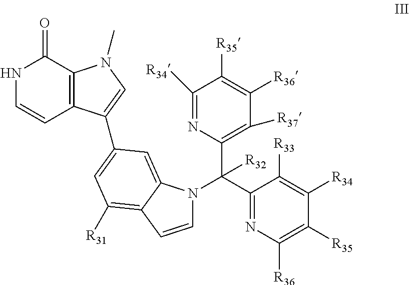

wherein R₃₁ is

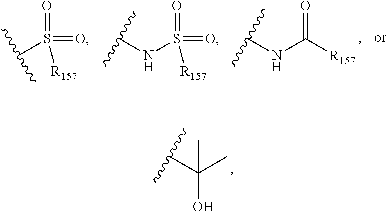

wherein R₁₅₇ is Me, CH₂CH₃, or CH(CH3)₂,
wherein R₃₂ is selected from the group consisting of: a C₁-C₆ alkyl, a C₁-C₆ alkenyl, a C₁-C₈ cycloalkyl, —H, -D, a C₁-C₈ substituted cycloalkylenyl, a substituted aryl, and a substituted heteroaryl, and
wherein R₃₃, R₃₄, R₃₅, R₃₆, R₃₇, R_{34′}, R_{35′}, R₃₆, R₃₇ are each independently selected from the group consisting of: —H, a halogen, —CN, C₁-C₃ haloalkyl, a C₁-C₆ cycloalkyl, a C₁-a C₆ alkylamine, a C₁-C₆ cycloalkylamine, a C₁-C₆ alkylester and a C₁-C₆ alkylamides.

In one aspect, R₃₃, R₃₄, R₃₅, R₃₆, R₃₇, R_{34′}, R_{35′}, R_{36′}, R_{37′} in formula III are each hydrogen. In another aspect, R₃₂ in formula III is C₁-C₃ alkyl such as, for example, methyl. In another aspect, R₃₁ in formula III is

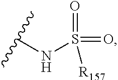

wherein R₁₅₇ is Me, CH₂CH₃, or CH(CH3)₂.

In another aspect, the compound is Formula IV

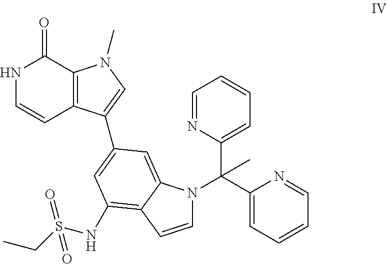 [SMILES: CCS(=O)(=O)Nc1cc(-c2cn(C)c3c(=O)[nH]ccc23)cc2c1ccn2C(C)(c1ccccn1)c1ccccn1]

or a pharmaceutically acceptable salt thereof. This compound is also referred to herein as XP-524.

Exemplary methods for producing compounds described herein are disclosed in WO 2019/109057, which is incorporated herein by reference in its entirety.

### Immune Checkpoint Inhibitors

In certain aspects, the pyridine-based compounds described herein can be co-administered with one or more immune checkpoint inhibitors. Immune checkpoint inhibitor therapy targets immune checkpoints, key regulators of the immune system that when stimulated can dampen the immune response to an immunologic stimulus. Some cancers can protect themselves from attack by stimulating immune checkpoint targets. Checkpoint therapy can block inhibitory checkpoints, restoring immune system function.

In one aspect, the immune checkpoint inhibitors include a PD-1 inhibitor, a PD-L1 inhibitor, or a CTLA-4 inhibitor. In one aspect, the immune checkpoint inhibitor is an antibody. In one aspect, the PD-1 inhibitor is Nivolumab (Opdivo®), Pembrolizumab (Keytruda®), and Cemiplimab (Libtayo®). In another aspect, the PD-L1 inhibitor is Atezolizumab (Tecentriq®), Avelumab (Bavencio®), and Durvalumab (Imfinzi®). In another aspect, the CTLA-4 inhibitor is Ipilimumab (Yervoy®).

In one aspect, this invention provides a method of treating PDAC comprising (a) administering to a subject in need of treatment an effective amount of a pyridine-based compound described herein; and (b) administering to the subject an effective amount of an immune checkpoint inhibitor. In another aspect, the administering of step (a) is prior to the administering of step (b). In another embodiment, the administering of step (a) is subsequent to the administering of step (b). In still another embodiment, the administering of step (a) is concurrent with the administering of step (b).

“Concurrent administration,” “administered in combination,” “simultaneous administration,” and similar phrases mean that two or more agents are administered concurrently to the subject being treated. By “concurrently,” it is meant that each agent is administered either simultaneously or sequentially in any order at different points in time. For example, a compound of formula I can be administered at the same time or sequentially in any order at different points in time as the immune checkpoint inhibitor. A compound of formula I the immune checkpoint inhibitor can be administered separately, in any appropriate form and by any suitable route. However, if not administered simultaneously, it is meant that they are administered to an individual in a sequence and sufficiently close in time so as to provide the desired therapeutic effect and can act in concert. When a compound of formula I and the immune checkpoint inhibitor are not administered concurrently, it is understood that they can be administered in any order to a subject in need thereof. For example, a compound of formula I can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of an immune checkpoint inhibitor, to an individual in need thereof. In various embodiments, a compound of formula I and an immune checkpoint inhibitor are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In one embodiment, the components of the combination therapies are administered at 1 minute to 24 hours apart.

In one aspect, the pyridine-based compound and immune checkpoint inhibitor are administered as separate unit doses. In another aspect, the pyridine-based compound and immune checkpoint inhibitor are formulated in a single pharmaceutical formulation with a pharmaceutically acceptable carrier.

### Pharmaceutical Compositions

In various aspects, the present disclosure relates to pharmaceutical compositions comprising a therapeutically effective amount of at least one disclosed compound, at least one product of a disclosed method, or a pharmaceutically acceptable salt thereof. As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

In a further aspect, the disclosed pharmaceutical compositions comprise a therapeutically effective amount of at least one disclosed compound, at least one product of a disclosed method, or a pharmaceutically acceptable salt thereof as an active ingredient, a pharmaceutically acceptable carrier, optionally one or more other therapeutic agent, and optionally one or more adjuvant. The disclosed pharmaceutical compositions include those suitable for oral, rectal, topical, pulmonary, nasal, and parenteral administration, although the most suitable route in any given case will depend on the particular host, and nature and severity of the conditions for which the active ingredient is being administered. In a further aspect, the disclosed pharmaceutical composition can be formulated to allow administration orally, nasally, via inhalation, parenterally, paracancerally, transmucosally, transdermally, intramuscularly, intravenously, intradermally, subcutaneously, intraperitoneally, intraventricularly, intracranially and intratumorally.

As used herein, “parenteral administration” includes administration by bolus injection or infusion, as well as administration by intravenous, intramuscular, intraarterial, intrathecal, intracapsular, intraorbital, intracardiac, intradermal, intraperitoneal, transtracheal, subcutaneous, subcuticular, intraarticular, subcapsular subarachnoid, intraspinal, epidural and intrasternal injection and infusion.

In various aspects, the present disclosure also relates to a pharmaceutical composition comprising a pharmaceutically acceptable carrier or diluent and, as active ingredient, a therapeutically effective amount of a disclosed compound, a product of a disclosed method of making, a pharmaceutically acceptable salt, a hydrate thereof, a solvate thereof, a polymorph thereof, or a stereochemically isomeric form thereof. In a further aspect, a disclosed compound, a product of a disclosed method of making, a pharmaceutically acceptable salt, a hydrate thereof, a solvate thereof, a polymorph thereof, or a stereochemically isomeric form thereof, or any subgroup or combination thereof may be formulated into various pharmaceutical forms for administration purposes.

In practice, the compounds of the present disclosure, or pharmaceutically acceptable salts thereof, of the present disclosure can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present disclosure can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the present disclosure, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

It is especially advantageous to formulate the aforementioned pharmaceutical compositions in unit dosage form for ease of administration and uniformity of dosage. The term “unit dosage form,” as used herein, refers to physically discrete units suitable as unitary dosages, each unit containing a predetermined quantity of active ingredient calculated to produce the desired therapeutic effect in association with the required pharmaceutical carrier. That is, a “unit dosage form” is taken to mean a single dose wherein all active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets (including scored or coated tablets), capsules or pills for oral administration; single dose vials for injectable solutions or suspension; suppositories for rectal administration; powder packets; wafers; and segregated multiples thereof. This list of unit dosage forms is not intended to be limiting in any way, but merely to represent typical examples of unit dosage forms.

The pharmaceutical compositions disclosed herein comprise a compound of the present disclosure (or pharmaceutically acceptable salts thereof) as an active ingredient, a pharmaceutically acceptable carrier, and optionally one or more additional therapeutic agents. In various aspects, the disclosed pharmaceutical compositions can include a pharmaceutically acceptable carrier and a disclosed compound, or a pharmaceutically acceptable salt thereof. In a further aspect, a disclosed compound, or pharmaceutically acceptable salt thereof, can also be included in a pharmaceutical composition in combination with one or more other therapeutically active compounds. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Techniques and compositions for making dosage forms useful for materials and methods described herein are described, for example, in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

The compounds described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for oral, rectal, topical, intravenous injection or parenteral administration. Carriers include solids or liquids, and the type of carrier is chosen based on the type of administration being used. The compounds may be administered as a dosage that has a known quantity of the compound.

Because of the ease in administration, oral administration can be a preferred dosage form, and tablets and capsules represent the most advantageous oral dosage unit forms in which case solid pharmaceutical carriers are obviously employed. However, other dosage forms may be suitable depending upon clinical population (e.g., age and severity of clinical condition), solubility properties of the specific disclosed compound used, and the like. Accordingly, the disclosed compounds can be used in oral dosage forms such as pills, powders, granules, elixirs, tinctures, suspensions, syrups, and emulsions. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

The disclosed pharmaceutical compositions in an oral dosage form can comprise one or more pharmaceutical excipient and/or additive. Non-limiting examples of suitable excipients and additives include gelatin, natural sugars such as raw sugar or lactose, lecithin, pectin, starches (for example corn starch or amylose), dextran, polyvinyl pyrrolidone, polyvinyl acetate, gum arabic, alginic acid, tylose, talcum, lycopodium, silica gel (for example colloidal), cellulose, cellulose derivatives (for example cellulose ethers in which the cellulose hydroxy groups are partially etherified with lower saturated aliphatic alcohols and/or lower saturated, aliphatic oxyalcohols, for example methyl oxypropyl cellulose, methyl cellulose, hydroxypropyl methyl cellulose, hydroxypropyl methyl cellulose phthalate), fatty acids as well as magnesium, calcium or aluminum salts of fatty acids with 12 to 22 carbon atoms, in particular saturated (for example stearates), emulsifiers, oils and fats, in particular vegetable (for example, peanut oil, castor oil, olive oil, sesame oil, cottonseed oil, corn oil, wheat germ oil, sunflower seed oil, cod liver oil, in each case also optionally hydrated); glycerol esters and polyglycerol esters of saturated fatty acids C₁₂H₂₄O₂ to C₁₈H₃₆O₂ and their mixtures, it being possible for the glycerol hydroxy groups to be totally or also only partly esterified (for example mono-, di- and triglycerides); pharmaceutically acceptable mono- or multivalent alcohols and polyglycols such as polyethylene glycol and derivatives thereof, esters of aliphatic saturated or unsaturated fatty acids (2 to 22 carbon atoms, in particular 10-18 carbon atoms) with monovalent aliphatic alcohols (1 to 20 carbon atoms) or multivalent alcohols such as glycols, glycerol, diethylene glycol, pentacrythritol, sorbitol, mannitol and the like, which may optionally also be etherified, esters of citric acid with primary alcohols, acetic acid, urea, benzyl benzoate, dioxolanes, glyceroformals, tetrahydrofurfuryl alcohol, polyglycol ethers with C1-C12-alcohols, dimethylacetamide, lactamides, lactates, ethyl carbonates, silicones (in particular medium-viscous polydimethyl siloxanes), calcium carbonate, sodium carbonate, calcium phosphate, sodium phosphate, magnesium carbonate and the like.

Other auxiliary substances useful in preparing an oral dosage form are those which cause disintegration (so-called disintegrants), such as: cross-linked polyvinyl pyrrolidone, sodium carboxymethyl starch, sodium carboxymethyl cellulose or microcrystalline cellulose. Conventional coating substances may also be used to produce the oral dosage form. Those that may for example be considered are: polymerizates as well as copolymerizates of acrylic acid and/or methacrylic acid and/or their esters; copolymerizates of acrylic and methacrylic acid esters with a lower ammonium group content (for example Eudragit® RS), copolymerizates of acrylic and methacrylic acid esters and trimethyl ammonium methacrylate (for example Eudragit® RL); polyvinyl acetate; fats, oils, waxes, fatty alcohols; hydroxypropyl methyl cellulose phthalate or acetate succinate; cellulose acetate phthalate, starch acetate phthalate as well as polyvinyl acetate phthalate, carboxy methyl cellulose; methyl cellulose phthalate, methyl cellulose succinate, -phthalate succinate as well as methyl cellulose phthalic acid half ester; zein; ethyl cellulose as well as ethyl cellulose succinate; shellac, gluten; ethylcarboxyethyl cellulose; ethacrylate-maleic acid anhydride copolymer; maleic acid anhydride-vinyl methyl ether copolymer; styrol-maleic acid copolymerizate; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; cellulose acetate succinate; polyarginine.

Plasticizing agents that may be considered as coating substances in the disclosed oral dosage forms are: citric and tartaric acid esters (acetyl-triethyl citrate, acetyl tributyl-, tributyl-, triethyl-citrate); glycerol and glycerol esters (glycerol diacetate, -triacetate, acetylated monoglycerides, castor oil); phthalic acid esters (dibutyl-, diamyl-, diethyl-, dimethyl-, dipropyl-phthalate), di-(2-methoxy- or 2-ethoxyethyl)-phthalate, ethylphthalyl glycolate, butylphthalylethyl glycolate and butylglycolate; alcohols (propylene glycol, polyethylene glycol of various chain lengths), adipates (diethyladipate, di-(2-methoxy- or 2-ethoxyethyl)-adipate; benzophenone; diethyl- and diburylsebacate, dibutylsuccinate, dibutyltartrate; diethylene glycol dipropionate; ethyleneglycol diacetate, -dibutyrate, -dipropionate; tributyl phosphate, tributyrin; polyethylene glycol sorbitan monooleate (polysorbates such as Polysorbar 50); sorbitan monooleate.

Moreover, suitable binders, lubricants, disintegrating agents, coloring agents, flavoring agents, flow-inducing agents, and melting agents may be included as carriers. The pharmaceutical carrier employed can be, for example, a solid, liquid, or gas. Examples of solid carriers include, but are not limited to, lactose, terra alba, sucrose, glucose, methylcellulose, dicalcium phosphate, calcium sulfate, mannitol, sorbitol talc, starch, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In various aspects, a binder can include, for example, starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. In a further aspect, a disintegrator can include, for example, starch, methyl cellulose, agar, bentonite, xanthan gum, and the like.

In various aspects, an oral dosage form, such as a solid dosage form, can comprise a disclosed compound that is attached to polymers as targetable drug carriers or as a prodrug. Suitable biodegradable polymers useful in achieving controlled release of a drug include, for example, polylactic acid, polyglycolic acid, copolymers of polylactic and polyglycolic acid, caprolactones, polyhydroxy butyric acid, polyorthoesters, polyacetals, polydihydropyrans, polycyanoacylates, and hydrogels, preferably covalently crosslinked hydrogels.

Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

A tablet containing a disclosed compound can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In various aspects, a solid oral dosage form, such as a tablet, can be coated with an enteric coating to prevent ready decomposition in the stomach. In various aspects, enteric coating agents include, but are not limited to, hydroxypropylmethylcellulose phthalate, methacrylic acid-methacrylic acid ester copolymer, polyvinyl acetate-phthalate and cellulose acetate phthalate. Akihiko Hasegawa “Application of solid dispersions of Nifedipine with enteric coating agent to prepare a sustained-release dosage form” Chem. Pharm. Bull. 33:1615-1619 (1985). Various enteric coating materials may be selected on the basis of testing to achieve an enteric coated dosage form designed ab initio to have a preferable combination of dissolution time, coating thicknesses and diametral crushing strength (e.g., see S. C. Porter et al. “The Properties of Enteric Tablet Coatings Made From Polyvinyl Acetate-phthalate and Cellulose acetate Phthalate”, J. Pharm. Pharmacol. 22:42p (1970)). In a further aspect, the enteric coating may comprise hydroxypropyl-methylcellulose phthalate, methacrylic acid-methacrylic acid ester copolymer, polyvinyl acetate-phthalate and cellulose acetate phthalate.

In various aspects, an oral dosage form can be a solid dispersion with a water soluble or a water insoluble carrier. Examples of water soluble or water insoluble carrier include, but are not limited to, polyethylene glycol, polyvinylpyrrolidone, hydroxypropylmethyl-cellulose, phosphatidylcholine, polyoxyethylene hydrogenated castor oil, hydroxypropylmethylcellulose phthalate, carboxymethylethylcellulose, or hydroxypropylmethylcellulose, ethyl cellulose, or stearic acid.

In various aspects, an oral dosage form can be in a liquid dosage form, including those that are ingested, or alternatively, administered as a mouth wash or gargle. For example, a liquid dosage form can include aqueous suspensions, which contain the active materials in admixture with excipients suitable for the manufacture of aqueous suspensions. In addition, oily suspensions may be formulated by suspending the active ingredient in a vegetable oil, for example arachis oil, olive oil, sesame oil or coconut oil, or in a mineral oil such as liquid paraffin. Oily suspensions may also contain various excipients. The pharmaceutical compositions of the present disclosure may also be in the form of oil-in-water emulsions, which may also contain excipients such as sweetening and flavoring agents.

For the preparation of solutions or suspensions it is, for example, possible to use water, particularly sterile water, or physiologically acceptable organic solvents, such as alcohols (ethanol, propanol, isopropanol, 1,2-propylene glycol, polyglycols and their derivatives, fatty alcohols, partial esters of glycerol), oils (for example peanut oil, olive oil, sesame oil, almond oil, sunflower oil, soya bean oil, castor oil, bovine hoof oil), paraffins, dimethyl sulfoxide, triglycerides and the like.

In the case of a liquid dosage form such as a drinkable solutions, the following substances may be used as stabilizers or solubilizers: lower aliphatic mono- and multivalent alcohols with 2-4 carbon atoms, such as ethanol, n-propanol, glycerol, polyethylene glycols with molecular weights between 200-600 (for example 1 to 40% aqueous solution), diethylene glycol monoethyl ether, 1,2-propylene glycol, organic amides, for example amides of aliphatic C1-C6-carboxylic acids with ammonia or primary, secondary or tertiary C1-C4-amines or C1-C4-hydroxy amines such as urea, urethane, acetamide, N-methyl acetamide, N,N-diethyl acetamide, N,N-dimethyl acetamide, lower aliphatic amines and diamines with 2-6 carbon atoms, such as ethylene diamine, hydroxyethyl theophylline, tromethamine (for example as 0.1 to 20% aqueous solution), aliphatic amino acids.

In preparing the disclosed liquid dosage form can comprise solubilizers and emulsifiers such as the following non-limiting examples can be used: polyvinyl pyrrolidone, sorbitan fatty acid esters such as sorbitan trioleate, phosphatides such as lecithin, acacia, tragacanth, polyoxyethylated sorbitan monooleate and other ethoxylated fatty acid esters of sorbitan, polyoxyethylated fats, polyoxyethylated oleotriglycerides, linolizated oleotriglycerides, polyethylene oxide condensation products of fatty alcohols, alkylphenols or fatty acids or also 1-methyl-3-(2-hydroxyethyl)imidazolidone-(2). In this context, polyoxyethylated means that the substances in question contain polyoxyethylene chains, the degree of polymerization of which generally lies between 2 and 40 and in particular between 10 and 20. Polyoxyethylated substances of this kind may for example be obtained by reaction of hydroxyl group-containing compounds (for example mono- or diglycerides or unsaturated compounds such as those containing oleic acid radicals) with ethylene oxide (for example 40 Mol ethylene oxide per 1 Mol glyceride). Examples of oleotriglycerides are olive oil, peanut oil, castor oil, sesame oil, cottonseed oil, corn oil. See also Dr. H. P. Fiedler “Lexikon der Hillsstoffe für Pharmazie, Kostnetik und angrenzende Gebiete” 1971, pages 191-195.

In various aspects, a liquid dosage form can further comprise preservatives, stabilizers, buffer substances, flavor correcting agents, sweeteners, colorants, antioxidants and complex formers and the like. Complex formers which may be for example be considered are: chelate formers such as ethylene diamine retrascetic acid, nitrilotriacetic acid, diethylene triamine pentacetic acid and their salts.

It may optionally be necessary to stabilize a liquid dosage form with physiologically acceptable bases or buffers to a pH range of approximately 6 to 9. Preference may be given to as neutral or weakly basic a pH value as possible (up to pH 8).

In order to enhance the solubility and/or the stability of a disclosed compound in a disclosed liquid dosage form, a parenteral injection form, or an intravenous injectable form, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also co-solvents such as alcohols may improve the solubility and/or the stability of the compounds according to the present disclosure in pharmaceutical compositions.

In various aspects, a disclosed liquid dosage form, a parenteral injection form, or an intravenous injectable form can further comprise liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Pharmaceutical compositions of the present disclosure suitable injection, such as parenteral administration, such as intravenous, intramuscular, or subcutaneous administration. Pharmaceutical compositions for injection can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for parenteral administration can include sterile aqueous or oleaginous solutions, suspensions, or dispersions. Furthermore, the compositions can be in the form of sterile powders for the extemporaneous preparation of such sterile injectable solutions or dispersions. In some aspects, the final injectable form is sterile and must be effectively fluid for use in a syringe. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. In some aspects, a disclosed parenteral formulation can comprise about 0.01-0.1 M, e.g. about 0.05 M, phosphate buffer. In a further aspect, a disclosed parenteral formulation can comprise about 0.9% saline.

In various aspects, a disclosed parenteral pharmaceutical composition can comprise pharmaceutically acceptable carriers such as aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but not limited to water, alcoholic/aqueous solutions, emulsions or suspensions, including saline and buffered media. Parenteral vehicles can include mannitol, normal serum albumin, sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like. In a further aspect, a disclosed parenteral pharmaceutical composition can comprise may contain minor amounts of additives such as substances that enhance isotonicity and chemical stability, e.g., buffers and preservatives. Also contemplated for injectable pharmaceutical compositions are solid form preparations that are intended to be converted, shortly before use, to liquid form preparations. Furthermore, other adjuvants can be included to render the formulation isotonic with the blood of the subject or patient.

In addition to the pharmaceutical compositions described herein above, the disclosed compounds can also be formulated as a depot preparation. Such long acting formulations can be administered by implantation (e.g., subcutaneously or intramuscularly) or by intramuscular injection. Thus, for example, the compounds can be formulated with suitable polymeric or hydrophobic materials (e.g., as an emulsion in an acceptable oil) or ion exchange resins, or as sparingly soluble derivatives, e.g., as a sparingly soluble salt.

Pharmaceutical compositions of the present disclosure can be in a form suitable for topical administration. As used herein, the phrase “topical application” means administration onto a biological surface, whereby the biological surface includes, for example, a skin area (e.g., hands, forearms, elbows, legs, face, nails, anus and genital areas) or a mucosal membrane. By selecting the appropriate carrier and optionally other ingredients that can be included in the composition, as is detailed herein below, the compositions of the present invention may be formulated into any form typically employed for topical application. A topical pharmaceutical composition can be in a form of a cream, an ointment, a paste, a gel, a lotion, milk, a suspension, an aerosol, a spray, foam, a dusting powder, a pad, and a patch. Further, the compositions can be in a form suitable for use in transdermal devices. These formulations can be prepared, utilizing a compound of the present disclosure, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot-on, as an ointment.

Ointments are semisolid preparations, typically based on petrolatum or petroleum derivatives. The specific ointment base to be used is one that provides for optimum delivery for the active agent chosen for a given formulation, and, preferably, provides for other desired characteristics as well (e.g., emollience). As with other carriers or vehicles, an ointment base should be inert, stable, nonirritating and nonsensitizing. As explained in Remington: The Science and Practice of Pharmacy, 19th Ed., Easton, Pa.: Mack Publishing Co. (1995), pp. 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight.

Lotions are preparations that are to be applied to the skin surface without friction. Lotions are typically liquid or semiliquid preparations in which solid particles, including the active agent, are present in a water or alcohol base. Lotions are typically preferred for treating large body areas, due to the ease of applying a more fluid composition. Lotions are typically suspensions of solids, and oftentimes comprise a liquid oily emulsion of the oil-in-water type. It is generally necessary that the insoluble matter in a lotion be finely divided. Lotions typically contain suspending agents to produce better dispersions as well as compounds useful for localizing and holding the active agent in contact with the skin, such as methylcellulose, sodium carboxymethyl-cellulose, and the like.

Creams are viscous liquids or semisolid emulsions, either oil-in-water or water-in-oil. Cream bases are typically water-washable, and contain an oil phase, an emulsifier and an aqueous phase. The oil phase, also called the “internal” phase, is generally comprised of petrolatum and/or a fatty alcohol such as cetyl or stearyl alcohol. The aqueous phase typically, although not necessarily, exceeds the oil phase in volume, and generally contains a humectant. The emulsifier in a cream formulation is generally a nonionic, anionic, cationic or amphoteric surfactant. Reference may be made to Remington: The Science and Practice of Pharmacy, supra, for further information.

Pastes are semisolid dosage forms in which the bioactive agent is suspended in a suitable base. Depending on the nature of the base, pastes are divided between fatty pastes or those made from a single-phase aqueous gel. The base in a fatty paste is generally petrolatum, hydrophilic petrolatum and the like. The pastes made from single-phase aqueous gels generally incorporate carboxymethylcellulose or the like as a base. Additional reference may be made to Remington: The Science and Practice of Pharmacy, for further information.

Gel formulations are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.e., gelling agents, are crosslinked acrylic acid polymers such as the family of carbomer polymers, e.g., carboxypolyalkylenes that may be obtained commercially under the trademark Carbopol™. Other types of preferred polymers in this context are hydrophilic polymers such as polyethylene oxides, polyoxyethylene-polyoxypropylene copolymers and polyvinylalcohol; modified cellulose, such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Sprays generally provide the active agent in an aqueous and/or alcoholic solution which can be misted onto the skin for delivery. Such sprays include those formulated to provide for concentration of the active agent solution at the site of administration following delivery, e.g., the spray solution can be primarily composed of alcohol or other like volatile liquid in which the active agent can be dissolved. Upon delivery to the skin, the carrier evaporates, leaving concentrated active agent at the site of administration.

Foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-a-can” formulation technique. Compositions thus formulated typically contain a low-boiling hydrocarbon, e.g., isopropane. Application and agitation of such a composition at the body temperature cause the isopropane to vaporize and generate the foam, in a manner similar to a pressurized aerosol foaming system. Foams can be water-based or aqueous alkanolic, but are typically formulated with high alcohol content which, upon application to the skin of a user, quickly evaporates, driving the active ingredient through the upper skin layers to the site of treatment.

Skin patches typically comprise a backing, to which a reservoir containing the active agent is attached. The reservoir can be, for example, a pad in which the active agent or composition is dispersed or soaked, or a liquid reservoir. Patches typically further include a frontal water permeable adhesive, which adheres and secures the device to the treated region. Silicone rubbers with self-adhesiveness can alternatively be used. In both cases, a protective permeable layer can be used to protect the adhesive side of the patch prior to its use. Skin patches may further comprise a removable cover, which serves for protecting it upon storage.

Examples of patch configuration which can be utilized with the present invention include a single-layer or multi-layer drug-in-adhesive systems which are characterized by the inclusion of the drug directly within the skin-contacting adhesive. In such a transdermal patch design, the adhesive not only serves to affix the patch to the skin, but also serves as the formulation foundation, containing the drug and all the excipients under a single backing film. In the multi-layer drug-in-adhesive patch a membrane is disposed between two distinct drug-in-adhesive layers or multiple drug-in-adhesive layers are incorporated under a single backing film.

Examples of pharmaceutically acceptable carriers that are suitable for pharmaceutical compositions for topical applications include carrier materials that are well-known for use in the cosmetic and medical arts as bases for e.g., emulsions, creams, aqueous solutions, oils, ointments, pastes, gels, lotions, milks, foams, suspensions, aerosols and the like, depending on the final form of the composition. Representative examples of suitable carriers according to the present invention therefore include, without limitation, water, liquid alcohols, liquid glycols, liquid polyalkylene glycols, liquid esters, liquid amides, liquid protein hydrolysates, liquid alkylated protein hydrolysates, liquid lanolin and lanolin derivatives, and like materials commonly employed in cosmetic and medicinal compositions. Other suitable carriers according to the present invention include, without limitation, alcohols, such as, for example, monohydric and polyhydric alcohols, e.g., ethanol, isopropanol, glycerol, sorbitol, 2-methoxyethanol, diethyleneglycol, ethylene glycol, hexyleneglycol, mannitol, and propylene glycol; ethers such as diethyl or dipropyl ether; polyethylene glycols and methoxypolyoxyethylenes (carbowaxes having molecular weight ranging from 200 to 20,000); polyoxyethylene glycerols, polyoxyethylene sorbitols, stearoyl diacetin, and the like.

Topical compositions of the present disclosure can, if desired, be presented in a pack or dispenser device, such as an FDA-approved kit, which may contain one or more unit dosage forms containing the active ingredient. The dispenser device may, for example, comprise a tube. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising the topical composition of the invention formulated in a pharmaceutically acceptable carrier may also be prepared, placed in an appropriate container, and labeled for treatment of an indicated condition.

Another patch system configuration which can be used by the present invention is a reservoir transdermal system design which is characterized by the inclusion of a liquid compartment containing a drug solution or suspension separated from the release liner by a semi-permeable membrane and adhesive. The adhesive component of this patch system can either be incorporated as a continuous layer between the membrane and the release liner or in a concentric configuration around the membrane. Yet another patch system configuration which can be utilized by the present invention is a matrix system design which is characterized by the inclusion of a semisolid matrix containing a drug solution or suspension which is in direct contact with the release liner. The component responsible for skin adhesion is incorporated in an overlay and forms a concentric configuration around the semisolid matrix.

Pharmaceutical compositions of the present disclosure can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

Pharmaceutical compositions containing a compound of the present disclosure, and/or pharmaceutically acceptable salts thereof, can also be prepared in powder or liquid concentrate form.

The pharmaceutical composition (or formulation) may be packaged in a variety of ways. Generally, an article for distribution includes a container that contains the pharmaceutical composition in an appropriate form. Suitable containers are well known to those skilled in the art and include materials such as bottles (plastic and glass), sachets, foil blister packs, and the like. The container may also include a tamper proof assemblage to prevent indiscreet access to the contents of the package. In addition, the container typically has deposited thereon a label that describes the contents of the container and any appropriate warnings or instructions.

The disclosed pharmaceutical compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Pharmaceutical compositions comprising a disclosed compound formulated in a compatible pharmaceutical carrier may also be prepared, placed in an appropriate container, and labeled for treatment of an indicated condition.

The exact dosage and frequency of administration depends on the particular disclosed compound, a product of a disclosed method of making, a pharmaceutically acceptable salt, solvate, or polymorph thereof, a hydrate thereof, a solvate thereof, a polymorph thereof, or a stereochemically isomeric form thereof; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compounds of the present disclosure.

The disclosed pharmaceutical compositions can further comprise other therapeutically active compounds, which are usually applied in the treatment of the above mentioned pathological or clinical conditions.

It is understood that the disclosed compositions can be prepared from the disclosed compounds. It is also understood that the disclosed compositions can be employed in the disclosed methods of using.

As already mentioned, the present disclosure relates to a pharmaceutical composition comprising a therapeutically effective amount of a disclosed compound, a product of a disclosed method of making, a pharmaceutically acceptable salt, a hydrate thereof, a solvate thereof, a polymorph thereof, and a pharmaceutically acceptable carrier. Additionally, the present disclosure relates to a process for preparing such a pharmaceutical composition, characterized in that a pharmaceutically acceptable carrier is intimately mixed with a therapeutically effective amount of a compound according to the present disclosure.

### Methods of Using the Pyridinone-Based Compounds and Formulations Thereof

Any amount of the pyridinone-based compounds and optional immune checkpoint inhibitor described herein can be administered to a subject in need thereof one or more times per day, week, month, or year. In some aspects, the amount administered is the effective amount of the pyridinone-based compound(s) and optional immune checkpoint inhibitor. For example, the pyridinone-based compound can be administered in a total daily dose. The total daily dose can be given in a single dose per day. In other aspects, the total daily dose can be administered over multiple doses per day, in which each dose can contain a fraction of the total daily dose to be administered (sub-doses). In some aspects, the amount of doses delivered per day can be 2, 3, 4, 5, 6 or more. In further aspects, the pyridinone-based compounds and optional immune checkpoint inhibitor can be administered to a subject one or more times per week, such as 1, 2, 3, 4, 5, or 6 times per week. In other aspects, the pyridinone-based compound and optional immune checkpoint inhibitor can be administered to a subject one or more times per month, such as 1, 2, 3, 4 to 5 or more times per month. In still further aspects, the pyridinone-based compounds and optional immune checkpoint inhibitor can be administered to a subject one or more times per year, such as 1, 2, 3, 4, 5, 6, 7, 8, 9, 10 to 11 or more times per year.

In some aspects, the pyridinone-based compounds and optional immune checkpoint inhibitor can be used as a co-therapy or combination therapy with one or more other auxiliary active agents or treatment modalities. In some aspects, the pyridinone-based compounds and optional immune checkpoint inhibitor can be administered in simultaneously with, contemporaneously with, and/or sequentially with a conventional chemotherapeutic agent or pharmaceutical formulation thereof, radiation, and/or other cancer treatment modality.

The amount of pyridinone-based compound and optional immune checkpoint inhibitor described herein can be administered in an amount ranging from about 0.001 mg to about 1000 mg per day, as calculated as the free or unsalted compound. In some aspects, the amount of pyridinone-based compound and optional immune checkpoint inhibitor can range from 0.001 mg/kg bodyweight to 1000 mg/kg bodyweight. In some aspects, the amount of pyridinone-based compound and optional immune checkpoint inhibitor can be about 0.1, 0.5, 1, 2, 3, 4, 5, 6, 7, 8, 9, 10, 11, 12, 13, 14, 15, 16, 17, 18, 19, 20, 25, 50, to about 100 mg/kg bodyweight. In some aspects, the amount administered is an effective amount when considered as a single dose or as a totality of sub-doses.

The pyridinone-based compound and optional immune checkpoint inhibitor can be administered in combinations with or include one or more other auxiliary agents or therapeutic compounds as discussed elsewhere herein. Suitable auxiliary agents include but are not limited to antisense or RNA interference molecules, chemotherapeutics, anti-neoplasic agents, hormones, antibiotics, antivirals, immunomodulating agents, anti-nausea, pain modifying compounds (such as opiates), anti-inflammatory agents, antipyretics, antibiotics, and/or antibodies or fragments thereof. The compound(s), and/or formulation(s), and/or additional therapeutic agent(s) can be administered simultaneously or sequentially by any convenient route in separate or combined pharmaceutical formulations. The additional therapeutic agents can be provided in their optically pure form or a pharmaceutically acceptable salt thereof.

### Kits

The pyridinone-based compounds and optional immune checkpoint inhibitor described herein can be presented as a combination kit. As used herein, the terms “combination kit” or “kit of parts” refers to the any of the compounds, derivatives thereof, or pharmaceutical formulations described herein, and any additional components that are used to package, sell, market, deliver, and/or administer the combination of elements or a single element, such as the primary active ingredient, contained therein. Such additional components include but are not limited to, packaging, syringes, blister packages, bottles, and the like. When one or more of the components (e.g. active agents) contained in the kit are administered simultaneously, the combination kit can contain the active agents in a single pharmaceutical formulation (e.g. a tablet) or in separate pharmaceutical formulations.

When the agents are not administered simultaneously, the combination kit can contain each agent in separate pharmaceutical formulations. The separate pharmaceutical formulations can be contained in a single package or in separate packages within the kit.

In some aspects, the combination kit also includes instructions printed on or otherwise contained in a tangible medium of expression. The instructions can provide information regarding the content of the compound or pharmaceutical formulations contained therein, safety information regarding the content of the compound(s) or pharmaceutical formulation(s) contained therein, information regarding the dosages, indications for use, and/or recommended treatment regimen(s) for the compound(s) and/or pharmaceutical formulations contained therein. In some aspects, the instructions provide directions for administering the compounds, pharmaceutical formulations, or salts thereof to a subject in need thereof. The subject in need thereof can have or be suspected of having a cancer or other disease that histone modifications can be modulated to treat and/or prevent a disease and/or a symptom thereof, which can include, but is not limited to, arthritis, lupus, pulmonary arterial hypertension, heart remodeling, and/or a neurodegenerative disease.

In some aspects, the kit can include one or more auxiliary active agents in addition to a pyridinone-based compound, derivative thereof, or a pharmaceutical formulation thereof. In some aspects the auxiliary active agent is a conventional chemotherapeutic agent or pharmaceutical formulation thereof. Chemotherapeutic agents can include busulfan, improsulfan, piposulfan, benzodepa, carboquone, meturedepa, uredepa, altretamine, triethylenemelamine, triethylenephosphoramide, triethylenethiophosphoramide, trimethylolomelamine, chlorambucil, chlornaphazine, cyclophosphamide, estramustine, ifosfamide, mechlorethamine, mechlorethamine oxide hydrochloride, melphalan, novembichin, phenesterine, prednimustine, trofosfamide, uracil mustard, carmustine, chlorozotocin, fotemustine, lomustine, nimustine, ranimustine, dacarbazine, mannomustine, mitobronitol, mitolactol, pipobroman, aclacinomycins, actinomycin F(1), anthramycin, azaserine, bleomycin, cactinomycin, carubicin, carzinophilin, chromomycin, dactinomycin, daunorubicin, daunomycin, 6-diazo-5-oxo-1-norleucine, doxorubicin, epirubicin, mitomycin C, mycophenolic acid, nogalamycin, olivomycin, peplomycin, plicamycin, porfiromycin, puromycin, streptonigrin, streptozocin, tubercidin, ubenimex, zinostatin, zorubicin, denopterin, methotrexate, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, fluororacil, tegafur, L-asparaginase, pulmozyme, aceglatone, aldophosphamide glycoside, aminolevulinic acid, amsacrine, bestrabucil, bisantrene, carboplatin, cisplatin, defofamide, demecolcine, diaziquone, elfornithine, elliptinium acetate, etoglucid, etoposide, flutamide, gallium nitrate, hydroxyurea, interferon-alpha, interferon-beta, interferon-gamma, interleukin-2, lentinan, lonidamine, mitoguazone, mitoxantrone, mopidamol, nitracrine, pentostatin, phenamet, pirarubicin, podophyllinic acid, 2-ethylhydrazide, procarbazine, razoxane, sizofiran, spirogermanium, paclitaxel, tamoxifen, teniposide, tenuazonic acid, triaziquone, 2,2′,2″-trichlorotriethylamine, urethan, vinblastine, vincristine, vindesine, and combinations thereof.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### ASPECTS

Aspect 1. A method of treating cancer associated with a Ras mutation in a subject in need of treatment comprising administering to the subject an effective amount of a compound of formula I or a pharmaceutically acceptable salt thereof

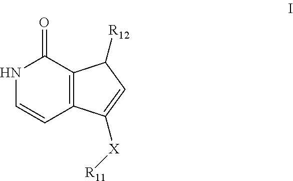

wherein
R₁₂ is a C₁-C₃ alkyl, a C₁-C₃ haloalkyl, propylenyl, —CH₂(CO)CH═CH₂, oxiran-2-ylmethyl, or CH₂(CO)CH₂Cl;
R₁₁ is a nitrogen-containing bicyclic or tricyclic heteroaryl, an aryl, or a biaryl, each of which is optionally substituted with 1, 2, or 3 substituents independently selected from —N(R^{a})S(O)₂R^{b}, —S(O)₂NR^{a}R^{b}, —C(O)NR^{a}R^{b}—N(R^{a})C(O)R^{b} —NR^{a}R^{b}, —(C₁-C₆ alkylenyl)R^{c}, —(C₁-C₃ cycloalkylenyl)R^{c}, aryl, heteroaryl, —(C₁-C₆ alkylenyl)R^{c}R^{c′}, —H, halogen, —CN, propylenyl, C₁-C₃ alkyl, C₁-C₃ haloalkyl, —OR₇₀, —NR₇₀R₇₀, —C(O)OR₇₀, —C(O)NR₇₀R₇₀, —S(O)₂R₇₀, —S(O)₂NR₇₀R₇₀, —CH₂(CO)CH═CH₂, oxiran-2-ylmethyl, CH₂(CO)CH₂Cl, and R₇₀;
X is optionally present, and when present, is selected from —O—, —C(O)—, —N(R₇₇)—, and —CH(R₇₀)—,
R₇₇ is selected from the group consisting of: —H, a halogen, —CN, C₁-C₃ haloalkyl, —OR₇₀, —NR₇₀R₇₀, —C(O)OR₇₀, —C(O)NR₇₀R₇₀, —S(O)₂R₇₀, —S(O)₂NR₇₀R₇₀, and R₇₀;
R₇₀, at each occurrence, are each independently selected from C₁-C₆ alkyl, C₂-C₆ alkenyl, C₂-C₆ alkynyl, halogen, C₁-C₆ haloalkyl, —CN, NO₂, —OR^{e}, —S(O)₂NR^{e}R^{f}, —C(O)R^{e}, —C(O)NR^{e}R^{f}, —NR^{e}R^{f} —N(R^{e})C(O)R^{f}, —(C₁-C₆ alkylenyl)-OR^{e}, —(C₁-C₆ alkylenyl)-C(O)NR^{e}R^{f}, a —(C₁-C₆alkylenyl)-NR^{e}R^{f}, and —(C₁-C₆ alkylenyl)-N(R^{e})C(O)R^{f};
R^{a} and R^{b}, at each occurrence, are independently selected from H, C₁-C₆ alkenyl, C₁-C₆ alkynyl, C₁-C₆ haloalkyl, R^{c}, and C₁-C₆ alkyl, wherein the C₁-C₆ alkyl is optionally substituted with one substituent selected from —OR^{e}, —NR^{e}R^{f}, —C(O)OR^{e}, —C(O)NR^{e}R^{f}, —S(O)₂R^{e}, —S(O)₂NR^{e}R^{f}, and R^{c};
R^{c} and R^{c′}, at each occurrence, are independently selected from aryl, heteroaryl, heterocycle, cycloalkyl, and cycloalkenyl, wherein each R^{c} group is optionally substituted with 1, 2, 3, 4, or 5 R^{d} groups;
R^{d}, at each occurrence, are independently selected from C₁-C₆ alkyl, C₂-C₆ alkenyl, C₂-C₆ alkynyl, halogen, C₁-C₆ haloalkyl, —CN, NO₂, —OR^{e}, —S(O)₂NR^{e}R^{f}, —C(O)R^{e}, —C(O)NR^{e}R^{f}, —NR^{e}R^{f}, —N(R^{e})C(O)R^{f}, —(C₁-C₆ alkylenyl)-OR^{e}, —(C₁-C₆ alkylenyl)-C(O)NR^{e}R^{f}, —(C₁-C₆ alkylenyl)-NR^{e}R^{f}, and —(C₁-C₆ alkylenyl)-N(R^{e})C(O)R^{f}; and
R^{e} and R^{f}, at each occurrence, are independently selected from H, C₁-C₆ alkyl, C₁-C₆ cycloalkyl, aryl, heteroaryl and C₁-C₆ haloalkyl.

Aspect 2. The method of Aspect 1, wherein the compound is Formula II

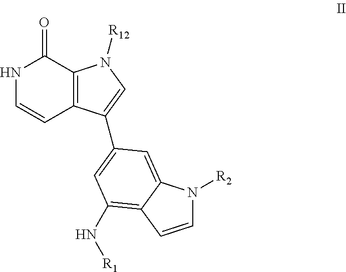

wherein R₁ is

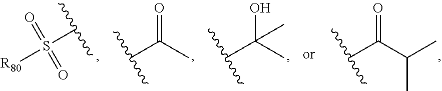

wherein R₂ is

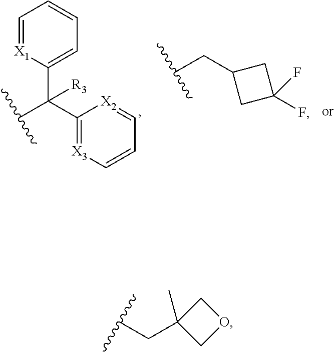

wherein R₃ is H or C₁-C₆ alkyl,
wherein R₈₀ is C₁-C₃ alkyl,
wherein X₁, X₂, and X₃, are each independently selected from the group consisting of: CH or N.

Aspect 3. The method of Aspects 1 or 2, wherein R₁₂ is C₁-C₃ alkyl.

Aspect 4. The method of any one of Aspects 1-3, wherein R₁₂ is methyl.

Aspect 5. The method of any one of Aspects 2-4, wherein R₂ is

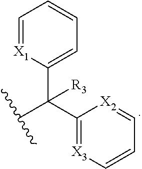

Aspect 6. The method of Aspect 5, wherein X₁ is N, X₂ is N, and X₃ is CH.

Aspect 7. The method of any one of Aspects 2-6, wherein R₃ is C₁-C₃ alkyl.

Aspect 8. The method of any one of Aspects 2-6, wherein R₃ is methyl.

Aspect 9. The method of any one of Aspects 2-8, wherein R₁ is

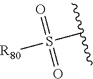

Aspect 10. The method of Aspect 9, wherein R₈₀ is ethyl.

Aspect 11. The method of Aspect 1, wherein the compound is Formula III

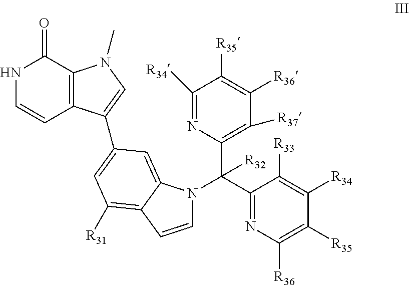

wherein R₃₁ is

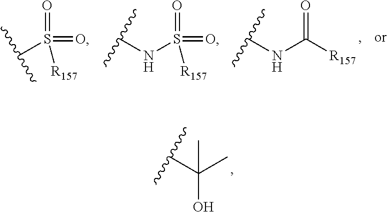

wherein R₁₅₇ is Me, CH₂CH₃, or CH(CH3)₂,
wherein R₃₂ is selected from the group consisting of: a C₁-C₆ alkyl, a C₁-C₆ alkenyl, a C₁-C₈ cycloalkyl, —H, -D, a C₁-C₈ substituted cycloalkylenyl, a substituted aryl, and a substituted heteroaryl, and wherein R₃₃, R₃₄, R₃₅, R₃₆, R₃₇, R_{34′}, R_{35′}, R₃₆, R₃₇ are each independently selected from the group consisting of: —H, a halogen, —CN, C₁-C₃ haloalkyl, a C₁-C₆ cycloalkyl, a C₁-a C₆ alkylamine, a C₁-C₆ cycloalkylamine, a C₁-C₆ alkylester and a C₁-C₆ alkylamides.

Aspect 12. The method of Aspect 11, wherein R₃₃, R₃₄, R₃₅, R₃₆, R₃₇, R_{34′}, R_{35′}, R₃₆, R₃₇ are each hydrogen.

Aspect 13. The method of Aspect 11 or 12, wherein R₃₂ is C₁-C₃ alkyl.

Aspect 14. The method of Aspect 11 or 12, wherein R₃₂ is methyl.

Aspect 15. The method of any one of Aspects 11-14, wherein R₃₁ is

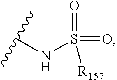

wherein R₁₅₇ is Me, CH₂CH₃, or CH(CH3)₂.

Aspect 16. The method of Aspect 1, wherein the compound is Formula IV

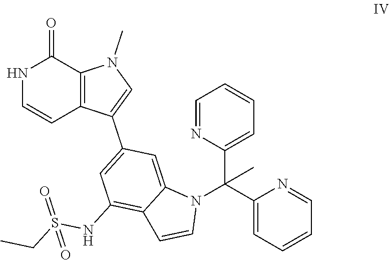 [SMILES: CCS(=O)(=O)Nc1cc(-c2cn(C)c3c(=O)[nH]ccc23)cc2c1ccn2C(C)(c1ccccn1)c1ccccn1]

or a pharmaceutically acceptable salt thereof.

Aspect 17. The method of any one of Aspects 1-16 further comprising administering to the subject an effective amount of an immune checkpoint inhibitor.

Aspect 18. The method of Aspect 17, wherein the immune checkpoint inhibitor comprises a PD-1 inhibitor, PD-L1 inhibitor, a CTLA-4 inhibitor, or a combination thereof.

Aspect 19. The method of Aspect 17 or 18, wherein the immune checkpoint inhibitor is anti-PD-1 antibody.

Aspect 20. The method of Aspect 17, wherein the immune checkpoint inhibitor comprises Nivolumab, Pembrolizumab, Cemiplimab, Atezolizumab, Avelumab, Durvalumab, or any combination thereof.

Aspect 21. The method of any one of Aspects 17-20, wherein the immune checkpoint inhibitor is administered prior to the administration of the compound.

Aspect 22. The method of any one of Aspects 17-20, wherein the immune checkpoint inhibitor is administered after the administration of the compound.

Aspect 23. The method of any one of Aspects 17-20, wherein the immune checkpoint inhibitor is administered concurrently with the administration of the compound.

Aspect 24. The method of any one of Aspects 1-23, wherein the cancer associated with a Ras mutation is lung cancer, gastrointestinal cancer, thoracic cancer, pancreatic cancer, colon cancer, or haematologic cancer.

Aspect 25. The method of any one of Aspects 1-23, wherein the cancer associated with a Ras mutation is small intestine adenocarcinoma, rectal adenocarcinoma, cholangiocarcinoma, gallbladder carcinoma, neuroblastoma, or melanoma.

Aspect 26. The method of any one of Aspects 1-23, wherein the cancer associated with a Ras mutation is pancreatic ductal adenocarcinoma.

Aspect 27. A pharmaceutical composition comprising
(a) a compound of formula I or a pharmaceutically acceptable salt thereof

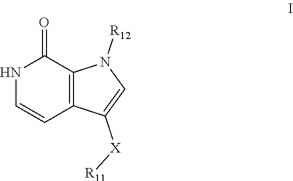

wherein
R₁₂ is a C₁-C₃ alkyl, a C₁-C₃ haloalkyl, propylenyl, —CH₂(CO)CH═CH₂, oxiran-2-ylmethyl, or CH₂(CO)CH₂Cl;
R₁₁ is a nitrogen-containing bicyclic or tricyclic heteroaryl, an aryl, or a biaryl, each of which is optionally substituted with 1, 2, or 3 substituents independently selected from —N(R^{a})S(O)₂R^{b}, —S(O)₂NR^{a}R^{b}, —C(O)NR^{a}R^{b}—N(R^{a})C(O)R^{b} —NR^{a}R^{b}, —(C₁-C₆ alkylenyl)R^{c}, —(C₁-C₃ cycloalkylenyl)R^{c}, aryl, heteroaryl, —(C₁-C₆ alkylenyl)R^{c}R^{c′}, —H, halogen, —CN, propylenyl, C₁-C₃ alkyl, C₁-C₃ haloalkyl, —OR₇₀, —NR₇₀R₇₀, —C(O)OR₇₀, —C(O)NR₇₀R₇₀, —S(O)₂R₇₀, —S(O)₂NR₇₀R₇₀, —CH₂(CO)CH═CH₂, oxiran-2-ylmethyl, CH₂(CO)CH₂Cl, and R₇₀;
X is optionally present, and when present, is selected from —O—, —C(O)—, —N(R₇₇)—, and —CH(R₇₀)—,
R₇₇ is selected from the group consisting of: —H, a halogen, —CN, C₁-C₃ haloalkyl, —OR₇₀, —NR₇₀R₇₀, —C(O)OR₇₀, —C(O)NR₇₀R₇₀, —S(O)₂R₇₀, —S(O)₂NR₇₀R₇₀, and R₇₀;
R₇₀, at each occurrence, are each independently selected from C₁-C₆ alkyl, C₂-C₆ alkenyl, C₂-C₆ alkynyl, halogen, C₁-C₆ haloalkyl, —CN, NO₂, —OR^{e}, —S(O)₂NR^{e}R^{f}, —C(O)R^{e}, —C(O)NR^{e}R^{f}, —NR^{e}R^{f} —N(R^{e})C(O)R^{f}, —(C₁-C₆ alkylenyl)-OR^{e}, —(C₁-C₆ alkylenyl)-C(O)NR^{e}R^{f}, a —(C₁-C₆alkylenyl)-NR^{e}R^{f}, and —(C₁-C₆ alkylenyl)-N(R^{e})C(O)R^{f};
R^{a} and R^{b}, at each occurrence, are independently selected from H, C₁-C₆ alkenyl, C₁-C₆ alkynyl, C₁-C₆ haloalkyl, R^{c}, and C₁-C₆ alkyl, wherein the C₁-C₆ alkyl is optionally substituted with one substituent selected from —OR^{e}, —NR^{e}R^{f}, —C(O)OR^{e}, —C(O)NR^{e}R^{f}, —S(O)₂R^{e}, —S(O)₂NR^{e}R^{f}, and R^{c};
R^{c} and R^{c′}, at each occurrence, are independently selected from aryl, heteroaryl, heterocycle, cycloalkyl, and cycloalkenyl, wherein each R^{c} group is optionally substituted with 1, 2, 3, 4, or 5 R^{d} groups;
R^{d}, at each occurrence, are independently selected from C₁-C₆ alkyl, C₂-C₆ alkenyl, C₂-C₆ alkynyl, halogen, C₁-C₆ haloalkyl, —CN, NO₂, —OR^{e}, —S(O)₂NR^{e}R^{f}, —C(O)R^{e}, —C(O)NR^{e}R^{f}, —NR^{e}R^{f}, —N(R^{e})C(O)R^{f}, —(C₁-C₆ alkylenyl)-OR^{e}, —(C₁-C₆ alkylenyl)-C(O)NR^{e}R^{f}, —(C₁-C₆ alkylenyl)-NR^{e}R^{f}, and —(C₁-C₆ alkylenyl)-N(R^{e})C(O)R^{f}; and
R^{e} and R^{f}, at each occurrence, are independently selected from H, C₁-C₆ alkyl, C₁-C₆ cycloalkyl, aryl, heteroaryl and C₁-C₆ haloalkyl;
(b) an immune checkpoint inhibitor; and
(c) a pharmaceutically acceptable carrier.

Aspect 28. A kit comprising
(a) a compound of formula I or a pharmaceutically acceptable salt thereof

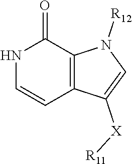

wherein
R₁₂ is a C₁-C₃ alkyl, a C₁-C₃ haloalkyl, propylenyl, —CH₂(CO)CH═CH₂, oxiran-2-ylmethyl, or CH₂(CO)CH₂Cl;
R₁₁ is a nitrogen-containing bicyclic or tricyclic heteroaryl, an aryl, or a biaryl, each of which is optionally substituted with 1, 2, or 3 substituents independently selected from —N(R^{a})S(O)₂R^{b}, —S(O)₂NR^{a}R^{b}, —C(O)NR^{a}R^{b}—N(R^{a})C(O)R^{b} —NR^{a}R^{b}, —(C₁-C₆ alkylenyl)R^{c}, —(C₁-C₃ cycloalkylenyl)R^{c}, aryl, heteroaryl, —(C₁-C₆ alkylenyl)R^{c}R^{c′}, —H, halogen, —CN, propylenyl, C₁-C₃alkyl, C₁-C₃haloalkyl, —OR₇₀, —NR₇₀R₇₀, —C(O)OR₇₀, —C(O)NR₇₀R₇₀, —S(O)₂R₇₀, —S(O)₂NR₇₀R₇₀, —CH₂(CO)CH═CH₂, oxiran-2-ylmethyl, CH₂(CO)CH₂Cl, and R₇₀;
X is optionally present, and when present, is selected from —O—, —C(O)—, —N(R₇₇)—, and —CH(R₇₀)—,
R₇₇ is selected from the group consisting of: —H, a halogen, —CN, C₁-C₃ haloalkyl, —OR₇₀, —NR₇₀R₇₀, —C(O)OR₇₀, —C(O)NR₇₀R₇₀, —S(O)₂R₇₀, —S(O)₂NR₇₀R₇₀, and R₇₀;
R₇₀, at each occurrence, are each independently selected from C₁-C₆ alkyl, C₂-C₆ alkenyl, C₂-C₆ alkynyl, halogen, C₁-C₆ haloalkyl, —CN, NO₂, —OR^{e}, —S(O)₂NR^{e}R^{f}, —C(O)R^{e}, —C(O)NR^{e}R^{f}, —NR^{e}R^{f} —N(R^{e})C(O)R^{f}, —(C₁-C₆ alkylenyl)-OR^{e}, —(C₁-C₆ alkylenyl)-C(O)NR^{e}R^{f}, a —(C₁-C₆alkylenyl)-NR^{e}R^{f}, and —(C₁-C₆ alkylenyl)-N(R^{e})C(O)R^{f};
R^{a} and R^{b}, at each occurrence, are independently selected from H, C₁-C₆ alkenyl, C₁-C₆ alkynyl, C₁-C₆ haloalkyl, R^{c}, and C₁-C₆ alkyl, wherein the C₁-C₆ alkyl is optionally substituted with one substituent selected from —OR^{e}, —NR^{e}R^{f}, —C(O)OR^{e}, —C(O)NR^{e}R^{f}, —S(O)₂R^{e}, —S(O)₂NR^{e}R^{f}, and R^{c};
R^{c} and R^{c′}, at each occurrence, are independently selected from aryl, heteroaryl, heterocycle, cycloalkyl, and cycloalkenyl, wherein each R^{c} group is optionally substituted with 1, 2, 3, 4, or 5 R^{d} groups;
R^{d}, at each occurrence, are independently selected from C₁-C₆ alkyl, C₂-C₆ alkenyl, C₂-C₆ alkynyl, halogen, C₁-C₆ haloalkyl, —CN, NO₂, —OR^{e}, —S(O)₂NR^{e}R^{f}, —C(O)R^{e}, —C(O)NR^{e}R^{f}, —NR^{e}R^{f}, —N(R^{e})C(O)R^{f}, —(C₁-C₆ alkylenyl)-OR^{e}, —(C₁-C₆ alkylenyl)-C(O)NR^{e}R^{f}, —(C₁-C₆ alkylenyl)-NR^{e}R^{f}, and —(C₁-C₆ alkylenyl)-N(R^{e})C(O)R^{f}; and
Rₑ and R^{f}, at each occurrence, are independently selected from H, C₁-C₆ alkyl, C₁-C₆ cycloalkyl, aryl, heteroaryl and C₁-C₆ haloalkyl;
(b) an immune checkpoint inhibitor; and
(c) instructions for administering the compound and the immune checkpoint inhibitor.

### EXAMPLES

The following examples are put forth so as to provide those of ordinary skill in the art with a complete disclosure and description of how the compounds, compositions, articles, devices and/or methods claimed herein are made and evaluated and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure.

### MATERIALS AND METHODS

### Cell Culture and Viability Assays

Human pancreatic cancer cells (Panc1 and MiaPaCa2) and murine pancreatic cancer cells (KPC105) were cultured in DMEM supplemented with 10% heat-inactivated FBS, penicillin (100 U/mL), and streptomycin (100 mg/mL). ASPC1 cells were grown in RPMI 1640 supplemented with 10% heat-inactivated FBS, penicillin (100U/mL), and streptomycin (100 mg/mL). Capan1 cells were grown in IMDM1 supplemented with 10% heat-inactivated FBS, penicillin (100U/mL), and streptomycin (100 mg/mL). Human cancer cell lines were purchased from the ATCC, used within six months, and kept under passage 10. KPC105 cells were used as described in our previous study (25). All cell lines in the laboratory were tested for mycoplasma every 6 months via LookOut Mycoplasma PCR Detection Kit (Sigma Aldrich) and used only if negative.

For cell growth assays, a fixed number of serum starved tumor cells were seeded into each well of a 24 well plate to reach 10% confluence, the subsequent percent confluence evaluated every 4 hours until the control group reached 100% confluence. For cell viability assays, 2,000-4,000 cells were seeded into each well of a 96-well plate in serum free DMEM. After 16 hours, media/drug was added and cells cultured for 72 hours. At this time, we added 20 ul of a 5 mg/ml 3-(4,5-Dimethyl-2-thiazolyl)-2,5-diphenyl-2H-tetrazolium bromide (MTT) solution to each well (1:100). After two hours, media was aspirated and crystals dissolved in DMSO and 570 nm absorbance determined by plate reader.

### Chemicals and Reagents

XP-524 was designed and synthesized as described previously (23). JQ-1 and SGC-CBP30 were purchased from Sigma Aldrich (St. Louis, MO) and reconstituted in sterile PBS at a stock concentration of 10 μM immediately before use.

### BROMOscan Bromodomain Profiling

BROMOscan bromodomain profiling was provided by DiscoverX Corp. (Fremont, CA) Kd of test compounds with DNA-tagged bromodomains and determined through binding against a proprietary reference immobilized ligand.

### Protein Crystallization

Crystals of the BRD4 and CBP bromodomains complexed with XP-524 were grown by hanging drop vapor diffusion at 4° C. Prior to crystallization, 16 mg/mL BRD4 or CBP bromodomain was incubated with 2 mM XP-524 for 60 min on ice and then centrifuged to remove precipitate. Crystals of the complex were grown by mixing 2 μL of BRD4: XP-524 or CBP: XP-524 with 1-2 μL of reservoir solution containing 17-19% PEG 3350 and 0.2 M lithium citrate tribasic tetrahydrate, pH 8.4.

### Data Collection and Structure Refinement

BRD4: XP-524 crystals were generated and analyzed as described previously (23). For CBP, crystals were cryo-preserved by soaking in mother liquor containing 10-15% glycerol and 200 μM of XP-524 before flash-freezing. Data were collected at the Life Sciences Collaborative Access Team 21-ID-F beamline at the Advanced Photon Source, Argonne National Laboratory. Data indexing, integration, and scaling were performed using XDS (74), and phases were determined by molecular replacement using first Phaser (75) and a CBP bromodomain structure (PDB entry: 5KTU) as search model. The assembly of four CBP:XP-524 monomers found by Phaser were then used as a search model by Molrep (76) to generate an asymmetric unit of two CBP:XP-524 “tetramers”. Rigid body refinement followed by iterative rounds of restrained refinement and model building were performed with CCP4i (77) modules Refmac5 (78) and Coot (79). The coordinates and structure factors have been deposited with PDB accession code 7JUO.

### Antibodies

All antibodies were purchased from established commercial vendors, and were verified by the manufacturer for the specific species and applications for which they were used in this manuscript.

### RNA Sequencing and Gene Set Enrichment Analysis (GSEA)

Panc1 cells were treated with either a DMSO vehicle, 1 μM of JQ-1, JQ-1 and SGC-CBP30, or XP-524. After 24 hours RNA was extracted using the RNeasy Plus Mini Kit (Qiagen, Hilden, Germany) per the manufacturer's instructions. Quality control, sequencing, and data analysis were performed by Novogene.

qPCR

Quantitative gene expression was performed with gene-specific TaqMan probes, TaqMan Universal PCR Master Mix, and the 7500 Fast Real-time PCR System from Applied Biosystems (Foster City, CA). These data were quantified with the comparative C_{T} method for relative gene expression as described in our previous study (70).

### Western Blot and Immunoprecipitation

Cell or tissue lysates were lysed in RIPA buffer (Cell Signaling) followed by sonication. Equal amounts of protein (15-50 μg) were mixed with loading dye, boiled for 8 min, separated on a 4-20% denaturing SDS-PAGE gel and transferred to a PVDF membrane. The membrane was blocked in 5% milk/TBS/0.1% Tween for one hour and incubated with antibodies against pRB, pMEK1, MEK1, pERK1/2, ERK1/2, (Cell Signaling, Danvers, MA), H3K27ac (Active Motif, Carlsbad, CA), KRAS (Novus Bio, Saint Charles, MO), BRD4, EP300 (abcam, Cambridge, MA), or GAPDH (Santa Cruz Biotech, Santa Cruz, CA). The membrane was washed with TBS-0.1% Tween and then incubated with HRP conjugated secondary antibody (Cell Signaling) at room temperature for one hour and rewashed. Protein bands were visualized by an enhanced chemiluminescence method (Thermo Fischer, Waltham, MA) and resolved digitally per the manufacturer's specifications.

For immunoprecipitation, cell lysates were collected using IP buffer (25 mM Tris-HCl pH 7.5, 150 mM NaCl, 1 mM EDTA, 0.1% NP-40 and 5% glycerol) with a protease and phosphatase inhibitory cocktail (Cell Signaling), and cell extracts were incubated overnight with the respective antibodies followed by incubation with protein A or G agarose beads for 4 h at 4° C. After washing 5-7 times with lysis buffer, immunocomplexes were resolved using SDS-PAGE and visualized by western blot. All antibodies were compared with isotype specific IgG controls to affirm specificity. All experiments were performed in triplicate unless otherwise specified.

### Transgenic Mice

Nongenic B6 (Wild Type), P48-Cre×LSL-Kras^{G12D} (KC), Pdx1-Cre×LSL-Kras^{G12D}×LSL-TP53^{R172H+/-} (KPC), and Pdx1-Cre×LSL-Kras^{G12D}×LSL-TP53^{R172H+/+} (KPPC) mice were generated as described previously (24). At 8 weeks of age, KC mice were administered an intraperitoneal injection (IP) of either a PBS vehicle or daily XP-524 (5 mg/kg). Mice were euthanized at 6 months of age and tissues collected for analysis. For studies involving KPC mice, animals were enrolled at 15 weeks of age, at which point they received IP injections of a PBS vehicle, XP-524 (5 mg/kg), anti-PD-1, or XP-524 and anti-PD-1 as described in the text. KPC mice were sacrificed when moribund or showing clear signs of health decline e.g. fur loss, weight loss, or lethargy, or for non-survival studies at the fixed endpoint shown in the number of days after enrollment. For studies involving KPPC mice, animals were enrolled either when developing a 0.5 cm, palpable tumor or at 4 weeks of age. At this time, mice were administered an intraperitoneal injection (IP) of either a PBS vehicle or daily XP-524 (5 mg/kg) and sacrificed when moribund or showing clear signs of health decline as described above. For euthanasia, animals were deeply anesthetized with isoflurane until unresponsive to toe tap and/or agonal breathing was observed. Thoracotomy served as the primary method of euthanasia and exsanguination the secondary method. For all mouse studies, mice were age matched within 2 weeks and males and females randomized at a 50:50 ratio.

### Primary Cell Line-Derived Xenografts

G-68 primary cell line-derived xenografts were generated as described previously (25). Also as described (25), tumor size was measured twice weekly with digital caliper. Mice were euthanized when moribund, when the maximum tumor size allowed per institutional policy (2 cm), or when tumors became ulcerated. For euthanasia, animals were scarified by CO₂ suffocation followed by cervical dislocation, and tumors subsequently harvested and processed as described above.

### PDAC Slice Cultures

Following the previously published protocol (29, 30), punch biopsies of −6 mm in diameter from human PDAC tumors were sectioned into 250 μm thick slices using the Leica VT1000S Vibrating blade microtome. The slices were then placed atop collagen-coated, 0.4 μm pore membrane inserts placed in 6-well plates. The next day, the slice cultures were treated with different drugs at the indicated doses. Fresh treatment media was replaced every 2-3 days. At the end of the experiment, slices were fixed with 4% PFA and processed by the Pathology Core at Northwestern University. The embedded tissues were subsequently embedded and sectioned for staining following the protocols described above.

### Histology, Immunohistochemistry, and Immunofluorescence

Mice were euthanized and the pancreas, colon, lungs, small bowel, liver, and spleen were subjected to pathologic examination. Tissues were fixed in 10% formalin, paraffin-embedded, and sections at 4 mm interval were cut from each tissue, and stained with hematoxylin and eosin (H&E), trichrome (Sigma Aldrich), or via immunohistochemistry (IHC) or immunofluorescence (IF). For immunohistochemistry, slides were deparaffinized by xylenes and rehydrated by ethanol gradient, then heated in a pressure cooker using DAKO retrieval buffer (DAKO, Santa Clara, CA). Endogenous peroxidases were quenched in 3% hydrogen peroxide in methanol for 30 minutes. Tissues were blocked with 0.5% BSA in PBS for 30 minutes and incubated with primary antibodies against: BRD4, EP300/CBP, CD45, CD8 (abcam), RAS^{G12D} (Genetex, Irvine, CA), pERK, or Cleaved Caspase 3 (Cell Signaling, Danvers, MA) at 1:50-1:200 overnight at 4° C. Slides were developed using HRP conjugated secondary antibodies followed by DAB substrate/buffer (DAKO).

For immunofluorescence, slides were heated via pressure cooker in DAKO retrieval buffer and tissues blocked with 0.5% BSA in PBS for 1 hour at room temperature. Sections were exposed to primary antibodies against CK19 (University of Iowa Hybridoma Bank), E-Cadherin (Cell Signaling), PCNA, CD3, (Santa Cruz Biotechnology, Dallas, TX), or Granzyme B (abcam, Cambridge, MA) at 1:50-1:200 overnight at 4° C. Slides were developed using AlexaFluor 488- or 594-conjugated secondary antibodies (1:200-1:1,000, Abcam), mounted in DAPI-containing media (Santa Cruz Biotechnology), exposed to DAPI, FITC, and Texas Red filters.

### Microscopy

All images were acquired using a Nikon 40×-400×Epi-Fluorescent Inverted Microscope with Phase Contrast Kit and Nikon bright field camera attachment. Negative slides were used for white balance, and for all images no analog or digital gain was used. For fluorescent imaging, we used positive control slides for each experiment and auto-exposed slides using Nikon NIS elements software using a gain setting of zero. Gain was similarly set to zero and LUTs were used to reduce background based on negative control slides. These LUT values and exposure times were standardized and used for all other similarly stained slides. Images were superimposed also using Nikon NIS elements software.

### Tissue Slide Counts and Measurements

All counts were performed by a minimum of three blinded, investigators and each value displays includes the average of at least three high power fields per specimen as described in our previous publications (24, 25, 67, 80-82). Score distributions were visualized via Minitab express software, showing the median value as a solid line and all individual values excluding any statistical outliers.

### Flow Cytometry

Spleens were isolated, ruptured, washed in cold PBS, and contents filtered. For tumor specimens, samples were digested with Collagenase IV/DNase at 37° C. for one hour, mechanically dissociated, and filtered at 100 μm. Two million cells were seeded into a round-bottom 96-well plate, washed in PBS, incubated with a Golgi plug/protein transport inhibitor (BD biosciences, San Jose, CA) and stained with Anti-CD45-FITC, Anti-CD11b-APC, Anti-CD206-PerCP/Cy5.5, Anti-GR-1-APC/Cy7 (BioLegend, San Diego, CA), Anti-CD4-APC/Cy7, Anti-CD25-PE, Ant-CD8A-PerCP/Cy5.5 (BD biosciences), Anti-CD69-APC, Anti-FoxP3-FITC (eBiosciences, San Diego, CA), anti-CD4-APC-Cy7, CD25-PE (BD biosciences), as well as an Alive/Dead kit (Invitrogen, Grand Island, NY) at 1:200-1:1500 in PBS over ice for 30 minutes. Cells were then fixed with 1% PFA in PBS for 10 minutes at room temperature, and for panels evaluating cytotoxic T-cells, stained with anti-Perforin-APC (Thermo Fischer) and anti-Interferon-γ-PE (BioLegend) at 1:500-150 in perm/stain buffer (BD biosciences) for 30 minutes over ice and washed three times with perm/wash buffer (BD biosciences). Analysis was conducted similarly using the same gating parameters and instrumentation, with a minimum of three mice included in each arm of analyses. Cells were analyzed with a BD Fortessa Cytometer, gating exclusively to cells within acceptable FSC/SSC parameters. All subsequent flow plots correspond to live, single cells based on Live/Dead assay and SSC-W gating, and are representative of 100,000 events unless otherwise stated. High and low populations were identified based on the geometric mean of the control group, based on unstained and isotype controls for each antibody. All other experiments were compared to both unstained, single cell, and isotype controls.

### Genomic Database Analysis

The Badea et al. and Pei et al. cohorts of PDAC patients were was downloaded and visualized using the using the Oncomine platform. Detailed information regarding these datasets and DNA/RNA sequencing analyses and protocols can be found on the data portal webpage listed above. All mRNA expression values are plotted in log scale unless otherwise noted.

### Authentication of Key Resources

All mouse genotypes were verified by tail biopsy followed by PCR for all relevant transgenes (Pdx1-Cre, P48-Cre, LSL-Kras^{G12D}, LSL-TP53^{R172H}). The anti-PD-1 antibody RMP1-14 is verified by the manufacturer (BioXcell) for the ability to recognize purified mouse PD-1 by western blot and has been successfully used to neutralize PD-1 signaling in murine pancreatic ductal adenocarcinoma (24, 83). Validation of the compound XP-524 is described in the manuscript text, as well as in the original work describing its synthesis (23).

### Statistical Analysis

Data were analyzed by either student's T test, simple linear regression analysis, hazard ratio, or ANOVA fit to a general linear model in Minitab express, the validity of which was tested by adherence to the normality assumption and the fitted plot of the residuals. Results were arranged by the Tukey method and considered significant at p<0.05 unless otherwise noted. Results are presented as either boxplot showing the median value and all other values arranged into quartiles, or as mean plus standard deviation. For RNA sequencing data, significance was determined using false discovery rate (FDR) adjusted P values and data considered significant at p<0.05.

### Results

### BRD4 and EP300/CBP are Ubiquitously Expressed in PDAC

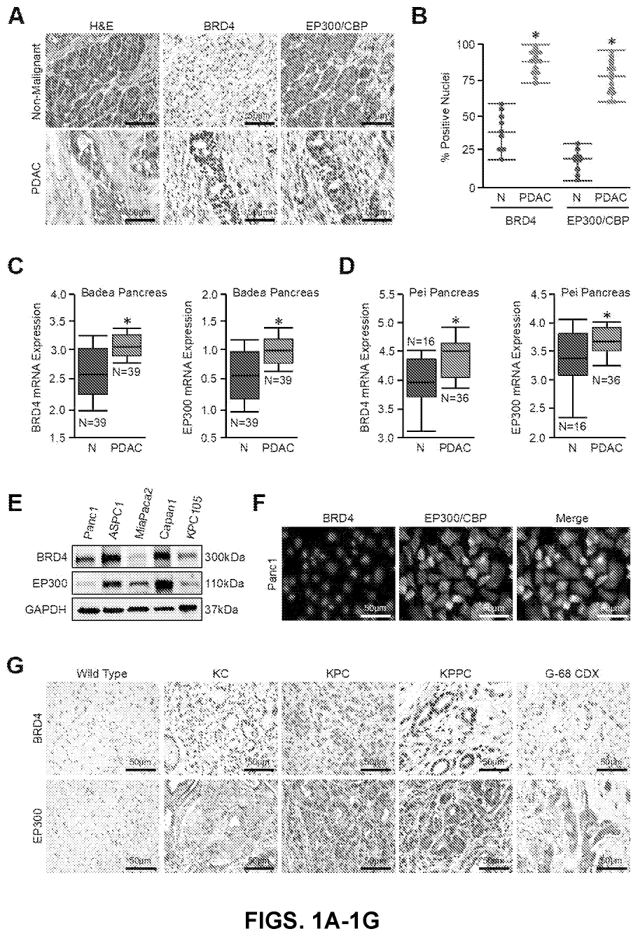

To determine the potential for a multi-specificity BRD4/EP300 inhibitor in PDAC, we first evaluated the expression of both proteins in a cohort of human PDAC excisional biopsies (N=14) and adjacent non-malignant tissue (N=9). In addition to an increase in staining intensity, PDAC tissues had a comparative increase in the percent of nuclei positive for both BRD4 and EP300/CBP when compared to adjacent non-malignant specimens (FIG. 1A,B). Similarly, both BRD4 and EP300 mRNA expression were increased in PDAC tumor specimens when compared to adjacent non-malignant tissues in two publicly available genomic datasets (FIG. 1C,D). BRD4 and EP300/CBP were ubiquitously expressed in PDAC cell lines (FIG. 1E), and displayed a similar pattern of nuclear localization in Panc1 cells, with exclusively nuclear expression of BRD4 and a combination of nuclear and cytoplasmic expression of EP300/CBP (FIG. 1F).

The expression of BRD4 and EP300/CBP was evaluated in the pancreata of increasingly aggressive mouse models of PDAC by immunohistochemistry. We observed a modest increase in nuclear accumulation of BRD4 and EP300/CBP in the Pdx1-Cre×LSL-Kras^{G12D/+} (KC) model of early disease, which localized predominantly to developing pancreatic epithelial neoplastic neoplasms (PanINs). We found stronger, uniform nuclear expression of both proteins in tumor tissue from the Pdx1-Cre×LSL-Kras^{G12D/+}×LSL-TP53^{R172H+/+} (KPC) model of invasive PDAC, as well as that of the Pdx1-Cre×LSL-Kras^{G12D/+}×LSL-TP53^{R172H+/+} (KPPC) model of extremely aggressive PDAC, and the G-68 primary cell line-derived xenograft (CDX) model (FIG. 1G).

XP-524 is a Potent, Multi-Specificity BET Inhibitor that Engages BRD4 and EP300/CBP

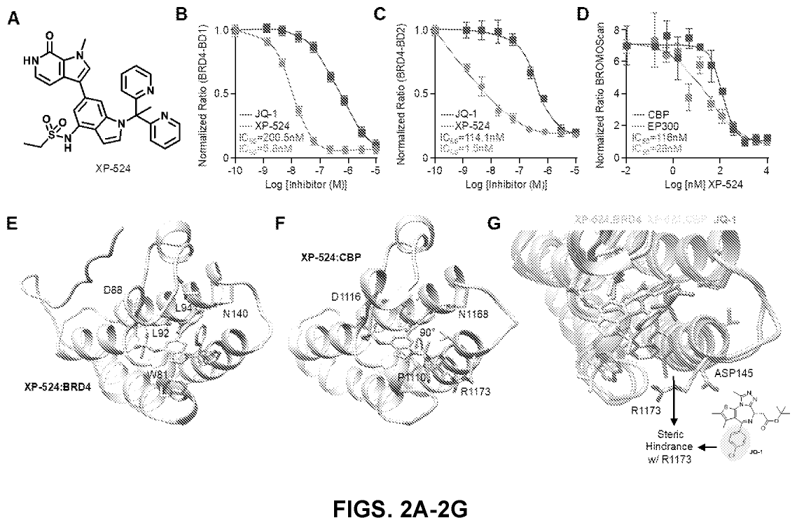

Given the conserved expression patterns of BRD4 and EP300/CBP in PDAC tissues, we next explored the potential of the novel compound XP-524 (23) as a dual-specificity inhibitor targeting the bromodomains of BRD4 and EP300/CBP (FIG. 2A). This compound showed superior potency compared to the prototypical, benchmark BET inhibitor JQ-1 by TR-FRET assay: XP-524 IC₅₀=5.8 nM (BRD4-BD1) 1.5 nM (BRD4-BD2); JQ-1 IC₅₀=200 nM (BRD4-BD1) 114 nM (BRD4-BD2) (FIG. 2B,C). Further analysis via the alternative BROMOscan assay confirmed that XP-524 binds strongly to and inhibits EP300 and CBP proteins with an IC₅₀ of 28 nM and 116 nM, respectively (FIG. 2D).

To verify the binding of XP-524 to its target proteins and explain the observed multi-specificity, we acquired co-crystal structures of XP-524 in complex with the bromodomains of BRD4 and the EP300 homolog CBP (CBP providing favorable solubility characteristics over EP300 for co-crystallization). We found that XP-524 similarly orients itself in both the BRD4-BD1 and the CBP bromodomain, making extensive contacts at the acetylated lysine pocket (FIG. 2E). The pyrrolopyridone group of XP-524 forms a bidentate hydrogen-bonding interaction with Asn-1168 in the CBP bromodomain, and the sulfate moiety inserts itself into the ZA loop and forms a hydrogen bond with the backbone of Asp 1116. The indole scaffold fits snugly into the LPF shelf through hydrophobic interactions (FIG. 2F).

An overlay of the XP-524-CBP complex with the XP-524-BRD4 complex demonstrated similar binding modes. The bottom pyridine ring of the CBP complex rotates 90° as CBP lacks the Trp for a pi-pi interaction in the LPF pocket. However, XP-524 forms a cation-pi interaction with Arg 1173, a key interaction in the design of potent EP300/CBP inhibitors (26-28). Contrastingly, JQ-1 is not able to capture this important cation-pi interaction, as the chlorophenyl ring of JQ-1 clashes with Arg-1173, thereby blocking the binding of JQ-1 to CBP and the ability of JQ-1 to act as an EP300/CBP inhibitor (FIG. 2G).

### XP-524 Disrupts Oncogenic KRAS Signaling In Vitro and Ex Vivo

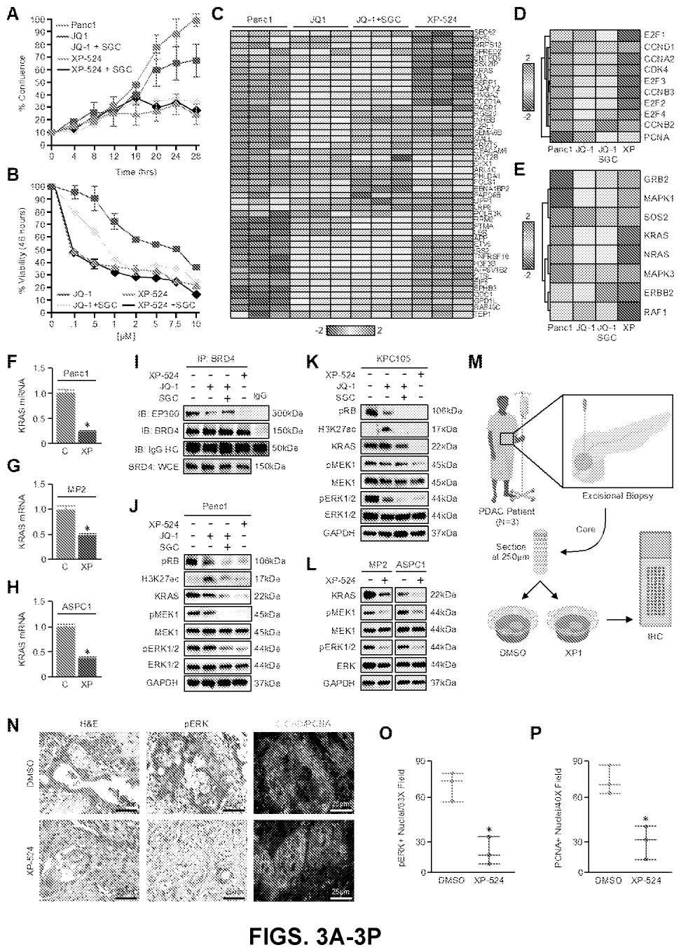

To explore the potential utility of the XP-524 compound in pancreatic cancer cells, we first incubated an equal number of Panc1 tumor cells with either a DMSO vehicle, 1 μM JQ-1, 1 μM of the EP300/CBP inhibitor SGC-CBP30, JQ-1 and SGC-CBP30, 1 μM XP-524, or XP-524 and SGC-CBP30, and monitored cell growth kinetics until DMSO-treated cells reached 100% confluence. While JQ-1 led to modest growth suppression, this was markedly enhanced by the addition of SGC-CBP30, though SGC-CBP30 had a marginal effect on cell growth without JQ-1 (FIG. 3A and S2A). Contrastingly, XP-524 was highly effective at suppressing tumor cell growth, closely resembling the combined effects of JQ-1 and SGC-CBP30. Consistent with its role as an EP300/CBP inhibitor, the effect of XP-524 was not further enhanced by the addition of SGC-CBP30 (FIG. 2A).

After repeating this experiment using MiaPaCa2 and ASPC1 cells (Figure S2B,C), we next incubated Panc1 cells with increasing concentrations of either JQ-1 or XP-524, with or without a fixed of 1 μM dose of SGC-CBP30. After 48 hours, we examined changes in cell viability via MTT assay. Similar to the effects observed on cell growth, the reduction in cell viability by JQ-1 was enhanced by the addition of SGC-CBP30, significantly reducing the observed IC50. While the combination of JQ-1 and SGC-CBP30 was similar in maximum efficacy to XP-524, XP-524 was more effective at lower concentrations and again unaffected by the addition of SGC-CBP30 (FIG. 3B and S2D).

To identify the cellular mechanisms that underlie these changes, we next incubated Panc1 tumor cells with either a DMSO vehicle, JQ-1, JQ-1 and SGC-CBP30, or XP-524. After 24 hours, cells were subjected to RNA sequencing (FIG. 3C). Post hoc gene set enrichment analysis suggested that XP-524 most significantly suppresses oncogenic KRAS signaling, as well as several associated cellular processes, including the MAPK pathway and cell cycle progression (FIG. 3D,E). While JQ-1 led to modest suppression of KRAS and cell cycle pathways, this was potentiated by the addition of SGC-CBP30, though less than the effects observed using XP-524 (FIG. 3D,E). Given the suggested effects of XP-524 on in KRAS mRNA by RNA sequencing, we next treated Panc1 cells as described and evaluated KRAS expression after 6 hours by qPCR. This affirmed a highly significant reduction in KRAS transcription following treatment with XP-524 (FIG. 3F), which was also observed using MiaPaCa2 and ASPC1 cells (FIG. 3G,H).

We then re-treated Panc1 cells with JQ-1, JQ-1 and SGC-CBP30, or XP-524 assessed alterations in protein expression by western blot. While JQ-1 and JQ-1/SGC-CBP30 modestly reduced co-precipitation of BRD4 and EP300/CBP compared to controls, this was nearly absent in cells incubated with XP-524 (FIG. 3I). This was associated with a significant reduction in KRAS expression as well as MEK/ERK activation following XP-524 treatment, paralleled by reductions in phosphorylated RB. Interestingly, JQ-1 treatment seemingly caused a compensatory increase in histone acetylation using H3K27ac as a surrogate marker, which was ameliorated with the addition of SGC-CBP30 and not observed in XP-524-treated cells (FIG. 3J). Using these same lysates, we also evaluated the association between BRD4 and EP300/CBP by immunoprecipitation. We next repeated this experiment using the primary KPC-105 murine PDAC cell line, which also demonstrated significant XP-524-induced suppression of KRAS expression and inhibition of downstream MEK/ERK activation, with similar results observed regarding RB phosphorylation and H3K27 acetylation (FIG. 3K). In more abbreviated experiments, XP-524 led to similar reductions in KRAS protein expression and downstream MEK/ERK activation in MiaPaCa2 and ASPC1 cells (FIG. 3L).

Given the inherent limitations of 2D cell culture, we next established primary PDAC slice cultures (FIG. 3M) by extracting 6 mm cores from resection specimens from 3 patients, sectioning cores at 250 μm intervals, and culturing ex vivo as described (29, 30). Slice cultures were incubated with either a DMSO vehicle or XP-524 (5 μM), and evaluated after 72 hours by immunohistochemistry. Consistent with our in vitro data (FIG. 3J), XP-524 significantly reduced ERK activation in human PDAC slice cultures, as well as cell proliferation in CK19+ tumor epithelial cells (FIG. 3N-P).

### XP-524 Reduces Mutant KRAS-Induced PanIN Formation In Vivo

Given the effects of XP-524 on disrupting oncogenic KRAS signaling in vitro, we next sought to determine whether XP-524 would similarly restrain KRAS-mediated oncogenesis in vivo. We, therefore, used the well-established Ptf1a-Cre×LSL-Kras^{G12D} (KC) model of neoplastic disease. This model serves as a reliable representation of early carcinogenesis, developing pancreatic intraepithelial neoplasms (PanINs) at 8 weeks that do not typically progress to invasive carcinoma (31, 32). This is well-represented in our colony, where KC mice are maintained in full a C57/B6 background and develop focal areas of PanIN disease at 8 weeks with 100% penetrance, which continue to develop throughout the pancreas with less than 5% of mice developing focal PDAC at one year of age.

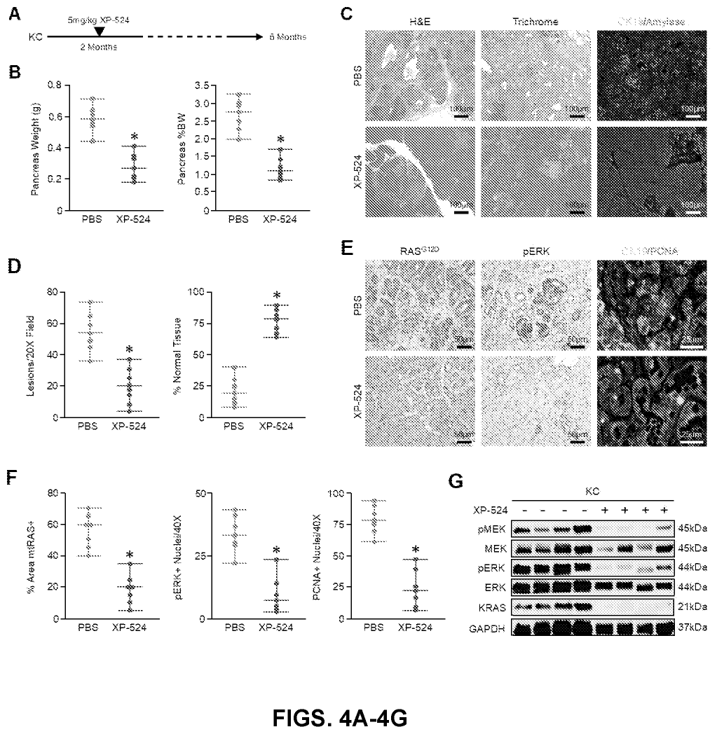

Using these mice, we first conducted a chemoprevention study in which 8-week-old KC animals were randomized at a 50:50 male to female ratio into one of two treatment groups. Mice were administered daily IP injections of either a PBS vehicle or 5 mg/kg XP-524, and sacrificed at a fixed time point of 6 months (FIG. 4A). Tissues were collected at the study endpoint, and mice treated with XP-524 showed a consistent reduction in the weight of the pancreas, particularly when normalized to total body weight (FIG. 4B). On histologic evaluation, XP-524-treated mice showed a significant reduction in lesion burden and fibrosis, as well as increased preservation of normal acinar tissue (FIG. 4C,D). Similar to results in cell culture systems, XP-524-treated mice had a substantial reduction in expression of the mutant RAS protein by IHC, paralleled by reductions in ERK activation and cell proliferation (FIG. 4E,F). This was also observed by western blotting, where XP-524-treated mice had a consistent reduction in KRAS expression, as well as downstream activation of MEK/ERK signals (FIG. 4G).

### XP-524 Extends Survival and Inhibits KRAS Signaling in Murine PDAC

To explore the efficacy of XP-524 in advanced disease, we next utilized two transgenic models of pancreatic carcinogenesis, the first being the Pdx1-Cre×LSL-Kras^{G12D}×LSL-TP53^{R172H+/-} (KPC) model of invasive PDAC. This model faithfully recapitulates human PDAC histotypes with several key features including poor immunogenicity and a dense, reactive tumor stroma. KPC animals were initially reported to develop precursor pancreatic intraepithelial neoplasms (PanIN) at 6 weeks, focal PDAC lesions at 15 weeks, and advanced PDAC at 18 weeks (33), though this can vary by individual colony and background strain. In our colony, KPC mice are maintained in full a C57/B6 background and have a mean survival of 124 days (N=30), with focal areas of PDAC developing at an average of 13 weeks of age (25).

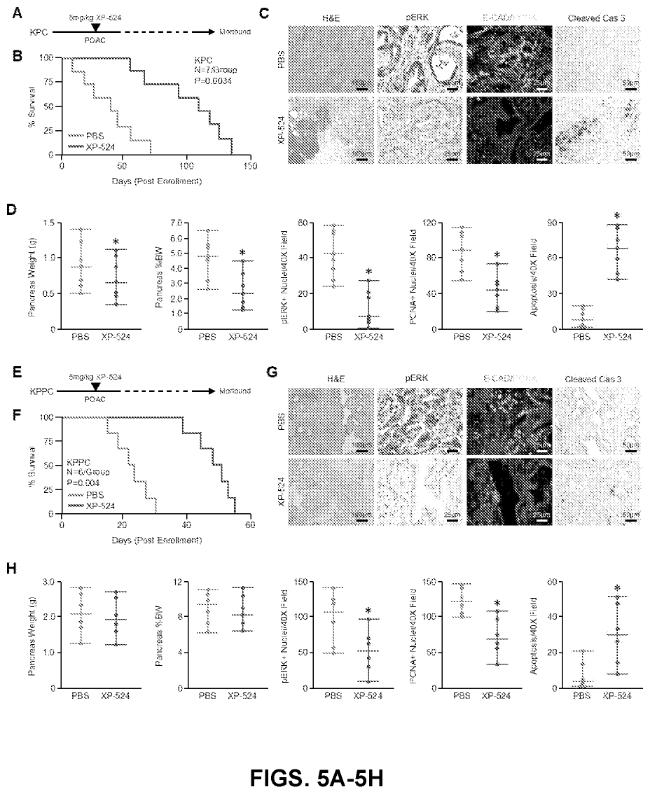

Using these mice, we first conducted an early intervention study in which mice were allowed to develop overt PDAC for a minimum of 15 weeks, at which point 100% of mice in our colony develop focal PDAC. At this time, mice were then randomized at a 50:50 male to female ratio into one of two treatment groups (N=7/group). Mice were administered daily IP injections of either a PBS vehicle or 5 mg/kg XP-524 and sacrificed when showing clear signs of health decline, e.g., weight loss, ascites, or lethargy (FIG. 5A). Interestingly, XP-524 significantly delayed mortality in KPC mice, extending median survival from 43 to 108 days post-enrollment (FIG. 5B). Tissues were collected at the study endpoint, sectioned, and stained either with H&E, trichrome, or immunohistochemistry for pERK. Consistent with our in vitro results, XP-524 treated mice had substantially reduced ERK activation, with parallel reductions in cell proliferation and uniform increases in apoptosis (FIG. 5C,D).

We next conducted a late intervention study using the Pdx1-Cre×LSL-Kras^{G12D}×LSL-TP53^{R172H+/+} (KPPC) model of extremely aggressive PDAC. In our colony, KPPC mice have a median survival of 43 days (N=12) and develop extensive PDAC as early as two weeks of age. We, therefore, enrolled mice as described either when they developed a 0.5 cm tumor or at 4 weeks of age and placed them into one of two treatment groups (N=6/group). As previously described, mice were administered daily IP injections of either a PBS vehicle or 5 mg/kg XP-524, and sacrificed when showing clear signs of health decline, e.g., weight loss, ascites, or lethargy (FIG. 5E). As in KPC mice, XP-524 delayed mortality in KPPC mice, extending median survival from 23 to 50 days post-enrollment (FIG. 5F). At the study endpoint, tissues were stained either with H&E or by immunohistochemistry, which revealed a modest reduction in tumor stroma and a significant decrease in ERK activation, and a subsequent increase in apoptosis (FIG. 5G,H).

### XP-524 Enhances T-Cell Recruitment but Fails to Promote a Functional Anti-Tumor Immune Response

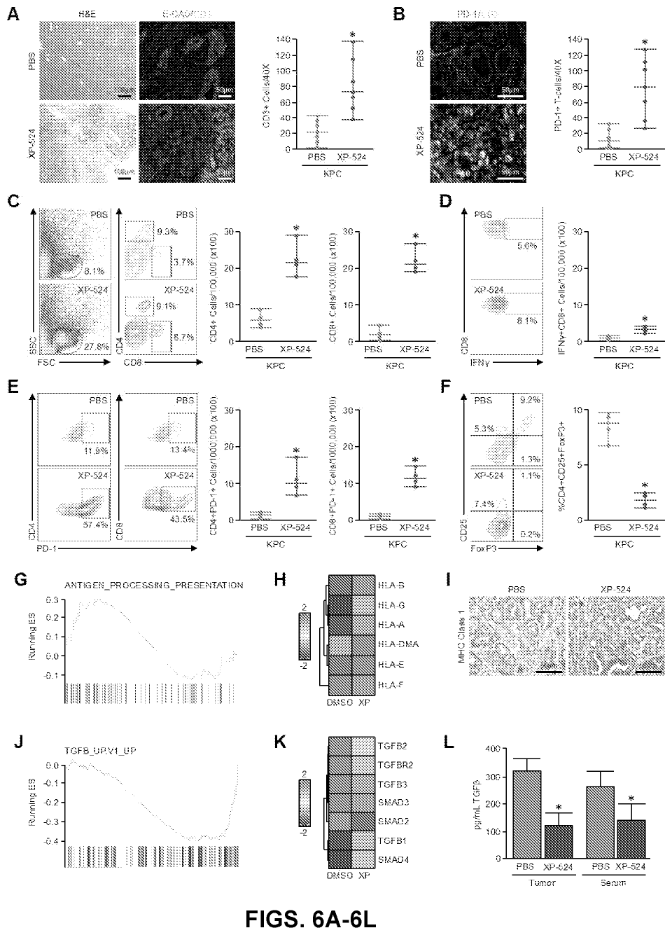

Beyond the described changes in mitosis and ERK activation, we also observed substantial changes in tumor architecture following treatment with XP-524. When compared to the KPC control group, XP-524 treated tumors had a highly cellular stroma, hallmarked by a consistent increase in lymphocyte infiltration, the majority of which stained positive for the T-cell marker CD3 and was confined to the tumor edge (FIG. 6A). However, these T-cells were largely positive for the T-cell exhaustion marker PD-1, and XP-524-treated tumors stained negatively for the surrogate marker of cytotoxicity GranzymeB (FIG. 6B and S3A). To better evaluate the immune infiltrate, we repeated our in vivo study, enrolling KPC mice as described and treating them for a fixed two-month period. At the study endpoint, the pancreata and spleens from PBS and XP-524 treated mice (N=4/group) were harvested and subjected to flow cytometry.

Consistent with the prior histopathology, tumors from XP-524-treated mice displayed a significant increase in the infiltration of both CD4+ and CD8+ T-cells (FIG. 6C), though there was no significant difference observed in the spleen (Figure S3B). However, when compared to positive controls, these intratumoral CD8+ T-cells were negative for the activation surrogate IFNγ (FIG. 6D), and both CD4+ and CD8+ T-cells were largely positive for PD-1 (FIG. 6E). Interestingly, XP-524-treated mice had a significant reduction in the relative abundance of intratumoral CD4+CD25+FoxP3+ regulatory T-cells (Tregs) (FIG. 6F), though this was not observed in the spleen (Figure S3B). Mice treated with XP-524 had no consistent increase in CD45+CD11b+GR-1+ macrophage infiltration, though we observed a modest decrease in expression of the M2 surrogate CD206 (Figure S3C).

To further explore mechanisms of XP-524-associated alterations in tumor immunogenicity, we revisited our previous RNA sequencing data in Panc1 cells and explored cell processes related to immune cell processes. Using this approach, we found that XP-524 led to upregulation of the antigen processing/presentation pathway (FIG. 6G), significantly increasing the mRNA expression of a variety of human leukocyte antigen (HLA) molecules (FIG. 6H). This was also observed in vivo, where XP-524 treated mice demonstrated increased surface staining for MHC Class 1 (FIG. 6I). RNA sequencing data also identified a pronounced suppression of the transforming growth factor β (TGFβ) pathway (FIG. 6J,K). We, therefore, evaluated the concentration of TGFβ1 by ELISA and found that KPC mice treated with XP-524 displayed reduced intratumoral and serum levels of TGFβ1 (FIG. 6L), also consistent with diminished peripheral tolerance.

XP-524 Cooperates with PD-1 Inhibition to Further Extend Survival in KPC Mice

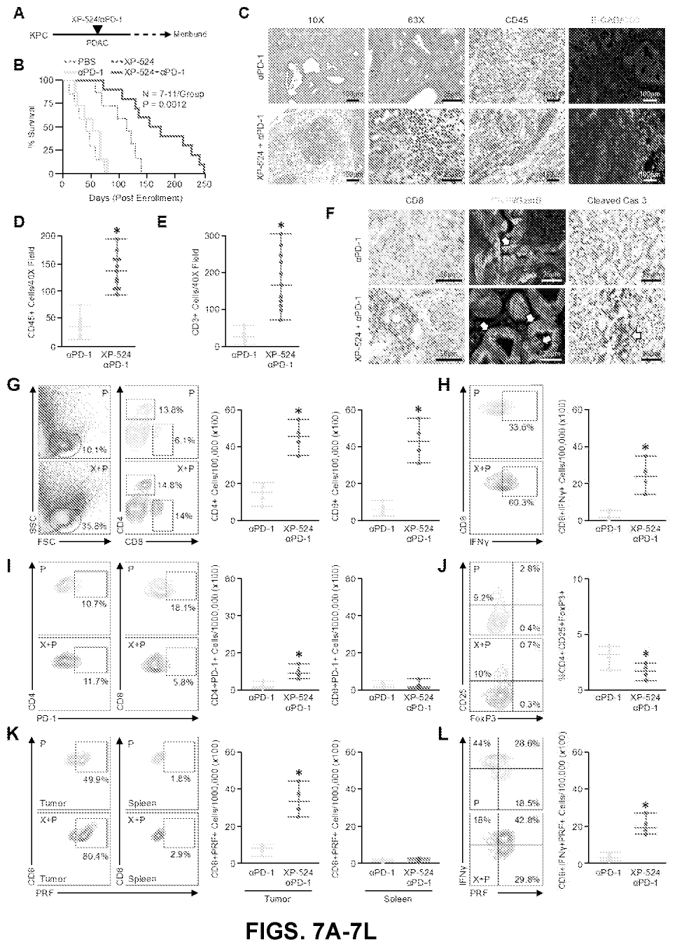

Given the enhanced infiltration of functionally exhausted T-cells in KPC mice treated with XP-524, as well as the observations that XP-524 increases antigen presentation and diminishes immunosuppressive TGFβ signaling, we next explored the combination of XP-524 with anti-PD-1 in vivo. KPC mice were again allowed to develop overt PDAC for a minimum of 14 weeks and randomized at a 50:50 male to female ratio into one of two treatment groups (N=7/group). Mice were administered either a fixed 200 μg dose of anti-PD-1 every other day and/or daily IP injections of 5 mg/kg XP-524 (FIG. 7A). While anti-PD-1 had no significant effect on survival, the combination of XP-524 and anti-PD-1 significantly improved survival, exceeding that observed with XP-524 alone. In contrast to the 51-day median survival observed with anti-PD-1 alone, mice treated with both XP-524 and anti-PD-1 had a median survival of 161 days post-enrollment (FIG. 7B). Tissues were again collected at the study endpoint, revealing a robust CD45+ infiltrate, paralleled by increased CD3+ T-cells (FIG. 7C-E). Additionally, we observed a highly significant increase in CD8+ T-cells, and the deposition of GranzymeB in remaining areas of disease accompanied by increased apoptosis (FIG. 7F).

We next repeated this experiment, enrolling KPC mice as described and treating with either anti-PD-1 or XP-524 and anti-PD-1 for two months. At the study endpoint, the pancreata and spleens from anti-PD-1 or XP-524 treated mice (N=4/group) were subjected to flow cytometry as described previously. While anti-PD-1 modestly increased the presence of tumor-infiltrating T-cells, mice treated with both drugs had enhanced infiltration of CD4+ and CD8+ T-cells, well surpassing that induced by XP-524 alone (FIG. 7G). However, unlike T-cells from mice treated with only XP-524, CD8+ T-cells were largely positive for IFNγ, and both CD4+ and CD8+ populations had low levels of the exhaustion marker PD-1 (FIG. 7H,I). As with XP-524 monotherapy, KPC mice treated with XP-524 and anti-PD-1 had a reduced frequency of intratumoral Tregs, CD4+CD25+FoxP3+ regulatory Tregs, with no change in splenic Tregs (FIG. 7J and S4A). The combination of XP-524 and anti-PD-1 led to a substantial increase in tumor infiltration of CD45+CD11b+GR-1+ macrophages, though a reduction in the percent of macrophages positive for the M2 marker CD206 (Figure S4B).

Consistent with an increase in cell-mediated cytotoxicity and reduction in T-cell exhaustion, the combination of XP-524 and anti-PD-1 enhanced expression of the surrogate marker of cytotoxicity Perforin-1 in tumor-infiltrating CD8+ T-cells, though no significant change was observed in splenic CD8+ T-cells (FIG. 7K). These Perforin-1-expressing CD8+ T-cells were also highly positive for the activation marker IFNγ, confirming the reduction in peripheral tolerance and increase in anti-tumor immune function following treatment with XP-524 and anti-PD-1 (FIG. 7L).

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. E. Hessmann, S. A. Johnsen, J. T. Siveke, V. Ellenrieder, Epigenetic treatment of pancreatic cancer: is there a therapeutic perspective on the horizon? Gut 66, 168-179 (2017).
2. A. Stathis, F. Bertoni, BET Proteins as Targets for Anticancer Treatment. Cancer Discov 8, 24-36 (2018).
3. V. Sahai, A. J. Redig, K. A. Collier, F. D. Eckerdt, H. G. Munshi, Targeting BET bromodomain proteins in solid tumors. Oncotarget 7, 53997-54009 (2016).
4. P. K. Mazur et al., Combined inhibition of BET family proteins and histone deacetylases as a potential epigenetics-based therapy for pancreatic ductal adenocarcinoma. Nat Med 21, 1163-1171 (2015).
5. V. Sahai et al., BET bromodomain inhibitors block growth of pancreatic cancer cells in three-dimensional collagen. Mol Cancer Ther 13, 1907-1917 (2014).
6. T. N. D. Pham et al., Induction of MNK Kinase-dependent eIF4E Phosphorylation by Inhibitors Targeting BET Proteins Limits Efficacy of BET Inhibitors. Mol Cancer Ther 18, 235-244 (2019).
7. K. Kumar et al., BET inhibitors block pancreatic stellate cell collagen I production and attenuate fibrosis in vivo. JCI Insight 2, e88032 (2017).
8. L. Zeng, M. M. Zhou, Bromodomain: an acetyl-lysine binding domain. FEBS Lett 513, 124-128 (2002).
9. A. C. Belkina, G. V. Denis, BET domain co-regulators in obesity, inflammation and cancer. Nat Rev Cancer 12, 465-477 (2012).
10. J. E. Delmore et al., BET bromodomain inhibition as a therapeutic strategy to target c-Myc. Cell 146, 904-917 (2011).
11. J. Loven et al., Selective inhibition of tumor oncogenes by disruption of super-enhancers. Cell 153, 320-334 (2013).
12. P. Filippakopoulos et al., Selective inhibition of BET bromodomains. Nature 468, 1067-1073 (2010).
13. H. Thomas, Therapy: Targeting chromatin remodelling proteins to treat pancreatic cancer. Nat Rev Gastroenterol Hepatol 12, 608 (2015).
14. J. Shi, C. R. Vakoc, The mechanisms behind the therapeutic activity of BET bromodomain inhibition. Mol Cell 54, 728-736 (2014).
15. G. Manzotti, A. Ciarrocchi, V. Sancisi, Inhibition of BET Proteins and Histone Deacetylase (HDACs): Crossing Roads in Cancer Therapy. Cancers (Basel) 11 (2019).
16. C. Dhalluin et al., Structure and ligand of a histone acetyltransferase bromodomain. Nature 399, 491-496 (1999).
17. D. A. Santillan et al., Bromodomain and histone acetyltransferase domain specificities control mixed lineage leukemia phenotype. Cancer Res 66, 10032-10039 (2006).
18. D. A. Hay et al., Discovery and optimization of small-molecule ligands for the CBP/p300 bromodomains. J Am Chem Soc 136, 9308-9319 (2014).
19. V. Garcia-Carpizo et al., CREBBP/EP300 bromodomains are critical to sustain the GATA1/MYC regulatory axis in proliferation. Epigenetics Chromatin 11, 30 (2018).
20. A. R. Conery et al., Bromodomain inhibition of the transcriptional coactivators CBP/EP300 as a therapeutic strategy to target the IRF4 network in multiple myeloma. Elife 5 (2016).
21. Y. Yan et al., The novel BET-CBP/p300 dual inhibitor NE02734 is active in SPOP mutant and wild-type prostate cancer. EMBO Mol Med 11, e10659 (2019).
22. X. Zhang et al., Therapeutic targeting of p300/CBP HAT domain for the treatment of NUT midline carcinoma. Oncogene 39, 4770-4779 (2020).
23. Y. Li et al., Novel Pyrrolopyridone Bromodomain and Extra-Terminal Motif (BET) Inhibitors Effective in Endocrine-Resistant ER+ Breast Cancer with Acquired Resistance to Fulvestrant and Palbociclib. J Med Chem 63, 7186-7210 (2020).
24. D. R. Principe et al., TGFbeta Blockade Augments PD-1 Inhibition to Promote T-Cell-Mediated Regression of Pancreatic Cancer. Mol Cancer Ther 18, 613-620 (2019).
25. D. R. Principe et al., Long-Term Gemcitabine Treatment Reshapes the Pancreatic Tumor Microenvironment and Sensitizes Murine Carcinoma to Combination Immunotherapy. Cancer Res 80, 3101-3115 (2020).
26. A. Hammitzsch et al., CBP30, a selective CBP/p300 bromodomain inhibitor, suppresses human Th17 responses. Proc Natl Acad Sci USA 112, 10768-10773 (2015).
27. W. A. Cortopassi, K. Kumar, R. S. Paton, Cation-pi interactions in CREBBP bromodomain inhibition: an electrostatic model for small-molecule binding affinity and selectivity. Org Biomol Chem 14, 10926-10938 (2016).
28. K. Kumar et al., Cation-pi interactions in protein-ligand binding: theory and data-mining reveal different roles for lysine and arginine. Chem Sci 9, 2655-2665 (2018).
29. X. Jiang, Y. D. Seo, K. M. Sullivan, V. G. Pillarisetty, Establishment of Slice Cultures as a Tool to Study the Cancer Immune Microenvironment. Methods Mol Biol 1884, 283-295 (2019).
30. C. Y. Lim et al., Organotypic slice cultures of pancreatic ductal adenocarcinoma preserve the tumor microenvironment and provide a platform for drug response. Pancreatology 18, 913-927 (2018).
31. C. B. Westphalen, K. P. Olive, Genetically engineered mouse models of pancreatic cancer. Cancer J 18, 502-510 (2012).
32. B. T. DeCant, D. R. Principe, C. Guerra, M. Pasca di Magliano, P. J. Grippo, Utilizing past and present mouse systems to engineer more relevant pancreatic cancer models. Front Physiol 5, 464 (2014).
33. S. R. Hingorani et al., Trp53R172H and KrasG12D cooperate to promote chromosomal instability and widely metastatic pancreatic ductal adenocarcinoma in mice. Cancer Cell 7, 469-483 (2005).
34. R. L. Siegel, K. D. Miller, A. Jemal, Cancer statistics, 2019. CA Cancer J Clin 69, 7-34 (2019).
35. P. R. Kunk, T. W. Bauer, C. L. Slingluff, O. E. Rahma, From bench to bedside a comprehensive review of pancreatic cancer immunotherapy. J Immunother Cancer 4, 14 (2016).
36. D. R. Principe, M. Korc, S. D. Kamath, H. G. Munshi, A. Rana, Trials and tribulations of pancreatic cancer immunotherapy. Cancer Lett 504, 1-14 (2021).
37. F. Xie et al., The BET inhibitor I-BET762 inhibits pancreatic ductal adenocarcinoma cell proliferation and enhances the therapeutic effect of gemcitabine. Sci Rep 8, 8102 (2018).
38. A. L. Miller et al., The BET inhibitor JQ1 attenuates double-strand break repair and sensitizes models of pancreatic ductal adenocarcinoma to PARP inhibitors. EBioMedicine 44, 419-430 (2019).
39. T. Wu, Y. F. Kamikawa, M. E. Donohoe, Brd4's Bromodomains Mediate Histone H3 Acetylation and Chromatin Remodeling in Pluripotent Cells through P300 and Brg1. Cell reports 25, 1756-1771 (2018).
40. H. Ono et al., C646 inhibits G2/M cell cycle-related proteins and potentiates anti-tumor effects in pancreatic cancer. Scientific Reports 11, 10078 (2021).
41. L. Buscail, B. Bournet, P. Cordelier, Role of oncogenic KRAS in the diagnosis, prognosis and treatment of pancreatic cancer. Nat Rev Gastroenterol Hepatol 17, 153-168 (2020).
42. M. P. di Magliano, C. D. Logsdon, Roles for KRAS in pancreatic tumor development and progression. Gastroenterology 144, 1220-1229 (2013).
43. V. T. Smit et al., KRAS codon 12 mutations occur very frequently in pancreatic adenocarcinomas. Nucleic Acids Res 16, 7773-7782 (1988).
44. C. Almoguera et al., Most human carcinomas of the exocrine pancreas contain mutant c-K-ras genes. Cell 53, 549-554 (1988).
45. M. A. Collins et al., Oncogenic Kras is required for both the initiation and maintenance of pancreatic cancer in mice. J Clin Invest 122, 639-653 (2012).
46. P. Liu, Y. Wang, X. Li, Targeting the untargetable KRAS in cancer therapy. Acta Pharm Sin B 9, 871-879 (2019).
47. D. S. Hong et al., CodeBreak 100: Phase I study of AMG 510, a novel KRASG12C inhibitor, in patients (pts) with advanced solid tumors other than non-small cell lung cancer NSCLC) and colorectal cancer (CRC). Journal of Clinical Oncology 38, 3511-3511 (2020).
48. T. Shimamura et al., Efficacy of BET bromodomain inhibition in Kras-mutant non-small cell lung cancer. Clin Cancer Res 19, 6183-6192 (2013).
49. S. A. Piha-Paul et al., First-in-Human Study of Mivebresib (ABBV-075), an Oral Pan-Inhibitor of Bromodomain and Extra Terminal Proteins, in Patients with Relapsed/Refractory Solid Tumors. Clin Cancer Res 25, 6309-6319 (2019).
50. K. Raina et al., PROTAC-induced BET protein degradation as a therapy for castration-resistant prostate cancer. Proc Natl Acad Sci USA 113, 7124-7129 (2016).
51. S. Postel-Vinay et al., First-in-human phase I study of the bromodomain and extraterminal motif inhibitor BAY 1238097: emerging pharmacokinetic/pharmacodynamic elationship and early termination due to unexpected toxicity. Eur J Cancer 109, 103-110 (2019).
52. P. J. O'Dwyer et al., Abstract CT014: GSK525762, a selective bromodomain (BRD) and extra terminal protein (BET) inhibitor: results from part 1 of a phase I/II open-abel single-agent study in patients with NUT midline carcinoma (NMC) and other cancers. Cancer Research 76, CT014-CT014 (2016).
53. A. J. Stonestrom et al., Functions of BET proteins in erythroid gene expression. Blood 125, 2825-2834 (2015).
54. S. A. Piha-Paul et al., Phase 1 Study of Molibresib (GSK525762), a Bromodomain and Extra-Terminal Domain Protein Inhibitor, in NUT Carcinoma and Other Solid Tumors. JNCI Cancer Spectr 4, pkz093 (2020).
55. J. Lewin et al., Phase Ib Trial With Birabresib, a Small-Molecule Inhibitor of Bromodomain and Extraterminal Proteins, in Patients With Selected Advanced Solid Tumors. J Clin Oncol 36, 3007-3014 (2018).
56. S. J. Nicholls et al., Efficacy and safety of a novel oral inducer of apolipoprotein a-I synthesis in statin-treated patients with stable coronary artery disease a randomized controlled trial. J Am Coll Cardiol 57, 1111-1119 (2011).
57. Y. Jiang, Y. Li, B. Zhu, T-cell exhaustion in the tumor microenvironment. Cell Death Dis 6, e1792 (2015).
58. F. S. Hodi et al., Improved survival with ipilimumab in patients with metastatic melanoma. N Engl J Med 363, 711-723 (2010).
59. C. Robert et al., Ipilimumab plus dacarbazine for previously untreated metastatic melanoma. N Engl J Med 364, 2517-2526 (2011).
60. H. Borghaei et al., Nivolumab versus Docetaxel in Advanced Nonsquamous Non-Small-Cell Lung Cancer. N Engl J Med 373, 1627-1639 (2015).
61. E. B. Garon et al., Pembrolizumab for the treatment of non-small-cell lung cancer. N Engl J Med 372, 2018-2028 (2015).
62. J. Larkin et al., Combined Nivolumab and Ipilimumab or Monotherapy in Untreated Melanoma. N Engl J Med 373, 23-34 (2015).
63. G. T. Gibney, L. M. Weiner, M. B. Atkins, Predictive biomarkers for checkpoint inhibitor-based immunotherapy. Lancet Oncol 17, e542-e551 (2016).
64. P. Darvin, S. M. Toor, V. Sasidharan Nair, E. Elkord, Immune checkpoint inhibitors: recent progress and potential biomarkers. Exp Mol Med 50, 1-11 (2018).
65. R. J. Torphy, Y. Zhu, R. D. Schulick, Immunotherapy for pancreatic cancer: Barriers and breakthroughs. Ann Gastroenterol Surg 2, 274-281 (2018).
66. J. Zhao et al., Irreversible electroporation reverses resistance to immune checkpoint blockade in pancreatic cancer. Nat Commun 10, 899 (2019).
67. D. R. Principe et al., TGFbeta Signaling in the Pancreatic Tumor Microenvironment Promotes Fibrosis and Immune Evasion to Facilitate Tumorigenesis. Cancer Res 76, 2525-2539 (2016).
68. H. Zhu et al., BET Bromodomain Inhibition Promotes Anti-tumor Immunity by Suppressing PD-L1 Expression. Cell Rep 16, 2829-2837 (2016).
69. S. J. Hogg et al., BET-Bromodomain Inhibitors Engage the Host Immune System and Regulate Expression of the Immune Checkpoint Ligand PD-L1. Cell Rep 18, 2162-2174 (2017).
70. K. Ebine et al., Interplay between interferon regulatory factor 1 and BRD4 in the regulation of PD-L1 in pancreatic stellate cells. Sci Rep 8, 13225 (2018).
71. D. O. Adeegbe et al., BET Bromodomain Inhibition Cooperates with PD-1 Blockade to Facilitate Antitumor Response in Kras-Mutant Non-Small Cell Lung Cancer. Cancer Immunol Res 6, 1234-1245 (2018).
72. Y. Pan et al., Synergistic inhibition of pancreatic cancer with anti-PD-L1 and c-Myc inhibitor JQ1. Oncoimmunology 8, e1581529 (2019).
73. J. A. Marin-Acevedo, R. M. Chirila, R. S. Dronca, Immune Checkpoint Inhibitor Toxicities. Mayo Clin Proc 94, 1321-1329 (2019).
74. W. Kabsch, Integration, scaling, space-group assignment and post-refinement. Acta Crystallogr D Biol Crystallogr 66, 133-144 (2010).
75. A. J. McCoy et al., Phaser crystallographic software. Journal of applied crystallography 40, 658-674 (2007).
76. A. Vagin, A. Teplyakov, Molecular replacement with MOLREP. Acta Crystallogr D Biol Crystallogr 66, 22-25 (2010).
77. E. Potterton, P. Briggs, M. Turkenburg, E. Dodson, A graphical user interface to the CCP4 program suite. Acta Crystallogr D Biol Crystallogr 59, 1131-1137 (2003).
78. G. N. Murshudov, A. A. Vagin, E. J. Dodson, Refinement of macromolecular structures by the maximum-likelihood method. Acta Crystallogr D Biol Crystallogr 53, 240-255 (1997).
79. P. Emsley, B. Lohkamp, W. G. Scott, K. Cowtan, Features and development of Coot. Acta Crystallogr D Biol Crystallogr 66, 486-501 (2010).
80. D. R. Principe et al., PEDF inhibits pancreatic tumorigenesis by attenuating the fibro-inflammatory reaction. Oncotarget 7, 28218-28234 (2016).
81. D. R. Principe et al., Loss of TGFbeta signaling promotes colon cancer progression and tumor-associated inflammation. Oncotarget 8, 3826-3839 (2017).
82. D. R. Principe et al., TGFbeta engages MEK/ERK to differentially regulate benign and malignant pancreas cell function. Oncogene 36, 4336-4348 (2017).
83. R. Winograd et al., Induction of T-cell Immunity Overcomes Complete Resistance to PD-1 and CTLA-4 Blockade and Improves Survival in Pancreatic Carcinoma. Cancer Immunol Res 3, 399-411 (2015).

1. A method of treating cancer associated with a Ras mutation in a subject in need of treatment comprising administering to the subject an effective amount of a compound of formula I or a pharmaceutically acceptable salt thereof 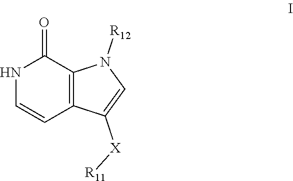
wherein
R₁₂ is a C₁-C₃ alkyl, a C₁-C₃ haloalkyl, propylenyl, —CH₂(CO)CH═CH₂, oxiran-2-ylmethyl, or CH₂(CO)CH₂Cl;
R₁₁ is a nitrogen-containing bicyclic or tricyclic heteroaryl, an aryl, or a biaryl, each of which is optionally substituted with 1, 2, or 3 substituents independently selected from —N(R^{a})S(O)₂R^{b}, —S(O)₂NR^{a}R^{b}, —C(O)NR^{a}R^{b}—N(R^{a})C(O)R^{b} —NR^{a}R^{b}, —(C₁-C₆ alkylenyl)R^{c}, —(C₁-C₃ cycloalkylenyl)R^{c}, aryl, heteroaryl, —(C₁-C₆ alkylenyl)R^{c}R^{c′}, —H, halogen, —CN, propylenyl, C₁-C₃ alkyl, C₁-C₃ haloalkyl, —OR₇₀, —NR₇₀R₇₀, —C(O)OR₇₀, —C(O)NR₇₀R₇₀, —S(O)₂R₇₀, —S(O)₂NR₇₀R₇₀, —CH₂(CO)CH═CH₂, oxiran-2-ylmethyl, CH₂(CO)CH₂Cl, and R₇₀;
X is optionally present, and when present, is selected from —O—, —C(O)—, —N(R₇₇)—, and —CH(R₇₀)—,
R₇₇ is selected from the group consisting of: —H, a halogen, —CN, C₁-C₃ haloalkyl, —OR₇₀, —NR₇₀R₇₀, —C(O)OR₇₀, —C(O)NR₇₀R₇₀, —S(O)₂R₇₀, —S(O)₂NR₇₀R₇₀, and R₇₀;
R₇₀, at each occurrence, are each independently selected from C₁-C₆ alkyl, C₂-C₆ alkenyl, C₂-C₆ alkynyl, halogen, C₁-C₆ haloalkyl, —CN, NO₂, —OR^{e}, —S(O)₂NR^{e}R^{f}, —C(O)R^{e}, —C(O)NR^{e}R^{f}, —NR^{e}R^{f} —N(R^{e})C(O)R^{f}, —(C₁-C₆ alkylenyl)-OR^{e}, —(C₁-C₆ alkylenyl)-C(O)NR^{e}R^{f}, a —(C₁-C₆alkylenyl)-NR^{e}R^{f}, and —(C₁-C₆ alkylenyl)-N(R^{e})C(O)R^{f};
R^{a} and R^{b}, at each occurrence, are independently selected from H, C₁-C₆ alkenyl, C₁-C₆ alkynyl, C₁-C₆ haloalkyl, R^{c}, and C₁-C₆ alkyl, wherein the C₁-C₆ alkyl is optionally substituted with one substituent selected from —OR^{e}, —NR^{e}R^{f}, —C(O)OR^{e}, —C(O)NR^{e}R^{f}, —S(O)₂R^{e}, —S(O)₂NR^{e}R^{f}, and R^{c};
R^{c} and R^{c′}, at each occurrence, are independently selected from aryl, heteroaryl, heterocycle, cycloalkyl, and cycloalkenyl, wherein each R^{c} group is optionally substituted with 1, 2, 3, 4, or 5 R^{d} groups;
R^{d}, at each occurrence, are independently selected from C₁-C₆ alkyl, C₂-C₆ alkenyl, C₂-C₆ alkynyl, halogen, C₁-C₆ haloalkyl, —CN, —NO₂, —OR^{e}, —S(O)₂NR^{e}R^{f}, —C(O)R^{e}, —C(O)NR^{e}R^{f}, —NR^{e}R^{f}, —N(R^{e})C(O)R^{f}, —(C₁-C₆ alkylenyl)-OR^{e}, —(C₁-C₆ alkylenyl)-C(O)NR^{e}R^{f}, —(C₁-C₆ alkylenyl)-NR^{e}R^{f}, and —(C₁-C₆ alkylenyl)-N(R^{e})C(O)R^{f}; and
R^{e} and R^{f}, at each occurrence, are independently selected from H, C₁-C₆ alkyl, C₁-C₆ cycloalkyl, aryl, heteroaryl and C₁-C₆ haloalkyl.

2. The method of claim 1, wherein the compound is Formula II 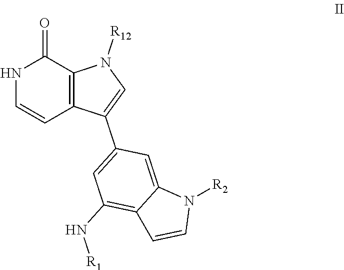
wherein R₁ is 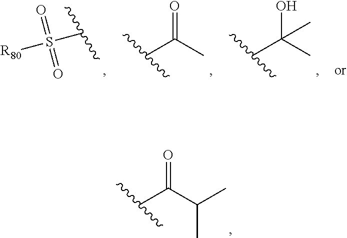
wherein R₂ is 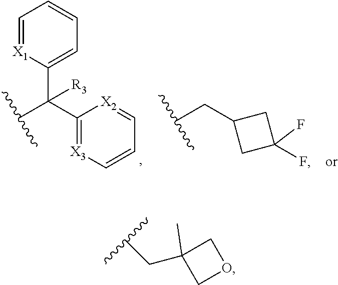
wherein R₃ is H or C₁-C₆ alkyl,
wherein R₈₀ is C₁-C₃ alkyl,
wherein X₁, X₂, and X₃, are each independently selected from the group consisting of: CH or N.

3. The method of claim 2, wherein R₁₂ is C₁-C₃ alkyl.

4. The method of claim 2, wherein R₁₂ is methyl.

5. The method of claim 2, wherein R₂ is

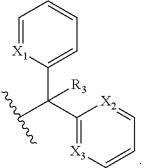

6. The method of claim 5, wherein X₁ is N, X₂ is N, and X₃ is CH.

7. The method of claim 5, wherein R₃ is C₁-C₃ alkyl.

8. The method of claim 5, wherein R₃ is methyl.

9. The method of claim 2, wherein R₁ is

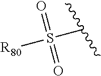

10. The method of claim 9, wherein R₈₀ is ethyl.

11. The method of claim 1, wherein the compound is Formula III 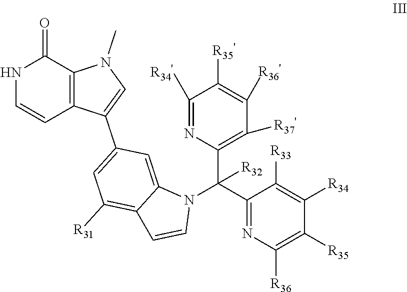
wherein R₃₁ is 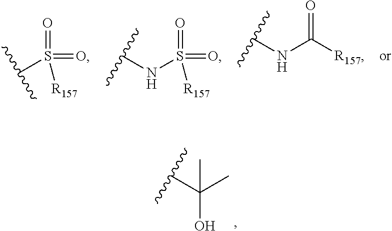
wherein R₁₅₇ is Me, CH₂CH₃, or CH(CH₃)₂,
wherein R₃₂ is selected from the group consisting of: a C₁-C₆ alkyl, a C₁-C₆ alkenyl, a C₁-C₈cycloalkyl, —H, -D, a C₁-C₆ substituted cycloalkylenyl, a substituted aryl, and a substituted heteroaryl,
and wherein R₃₃, R₃₄, R₃₅, R₃₆, R₃₇, R_{34′}, R_{35′}, R_{36′}, R_{37′} are each independently selected from the group consisting of: —H, a halogen, —CN, C₁-C₃ haloalkyl, a C₁-C₆ cycloalkyl, a C₁-a C₆ alkylamine, a C₁-C₆ cycloalkylamine, a C₁-C₆ alkylester and a C₁-C₆ alkylamides.

12. The method of claim 11, wherein R₃₃, R₃₄, R₃₅, R₃₆, R₃₇, R_{34′}, R_{35′}, R_{36′}, R_{37′} are each hydrogen.

13. The method of claim 11, wherein R₃₂ is C₁-C₃ alkyl.

14. The method of claim 11, wherein R₃₂ is methyl.

15. The method of claim 11, wherein R₃₁ is 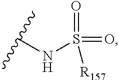
wherein R₁₅₇ is Me, CH₂CH₃, or CH(CH3)₂.

16. The method of claim 1, wherein the compound is Formula IV 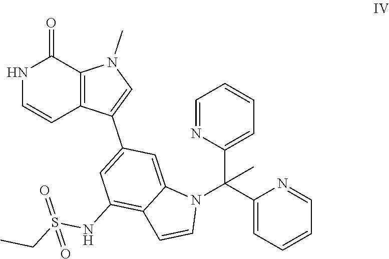 [SMILES: CCS(=O)(=O)Nc1cc(-c2cn(C)c3c(=O)[nH]ccc23)cc2c1ccn2C(C)(c1ccccn1)c1ccccn1]
or a pharmaceutically acceptable salt thereof.

17. The method of claim 1 further comprising administering to the subject an effective amount of an immune checkpoint inhibitor.

18. The method of claim 17, wherein the immune checkpoint inhibitor comprises a PD-1 inhibitor, PD-L1 inhibitor, a CTLA-4 inhibitor, an anti-PD-1 antibody, Nivolumab, Pembrolizumab, Cemiplimab, Atezolizumab, Avelumab, Durvalumab, or a combination thereof.

19. (canceled)

20. (canceled)

21. (canceled)

22. (canceled)

23. (canceled)

24. The method of claim 1, wherein the cancer associated with a Ras mutation is lung cancer, gastrointestinal cancer, thoracic cancer, pancreatic cancer, colon cancer, haematologic cancer, or cancer associated with a Ras mutation selected from the group consisting of small intestine adenocarcinoma, rectal adenocarcinoma, cholangiocarcinoma, gallbladder carcinoma, neuroblastoma, melanoma, head and neck squamous cell carcinoma, and pancreatic ductal adenocarcinoma.

25. (canceled)

26. (canceled)

27. A pharmaceutical composition comprising
(a) a compound of formula I or a pharmaceutically acceptable salt thereof 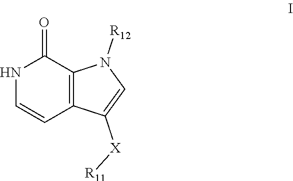
wherein
R₁₂ is a C₁-C₃ alkyl, a C₁-C₃ haloalkyl, propylenyl, —CH₂(CO)CH═CH₂, oxiran-2-ylmethyl, or CH₂(CO)CH₂Cl;
R₁₁ is a nitrogen-containing bicyclic or tricyclic heteroaryl, an aryl, or a biaryl, each of which is optionally substituted with 1, 2, or 3 substituents independently selected from —N(R^{a})S(O)₂R^{b}, —S(O)₂NR^{a}R^{b}, —C(O)NR^{a}R^{b}—N(R^{a})C(O)R^{b} —NR^{a}R^{b}, —(C₁-C₆ alkylenyl)R^{c}, —(C₁-C₃ cycloalkylenyl)R^{c}, aryl, heteroaryl, —(C₁-C₆ alkylenyl)R^{c}R^{c′}, —H, halogen, —CN, propylenyl, C₁-C₃ alkyl, C₁-C₃ haloalkyl, —OR₇₀, —NR₇₀R₇₀, —C(O)OR₇₀, —C(O)NR₇₀R₇₀, —S(O)₂R₇₀, —S(O)₂NR₇₀R₇₀, —CH₂(CO)CH═CH₂, oxiran-2-ylmethyl, CH₂(CO)CH₂Cl, and R₇₀;
X is optionally present, and when present, is selected from —O—, —C(O)—, —N(R₇₇)—, and —CH(R₇₀)—,
R₇₇ is selected from the group consisting of: —H, a halogen, —CN, C₁-C₃ haloalkyl, —OR₇₀, —NR₇₀R₇₀, —C(O)OR₇₀, —C(O)NR₇₀R₇₀, —S(O)₂R₇₀, —S(O)₂NR₇₀R₇₀, and R₇₀;
R₇₀, at each occurrence, are each independently selected from C₁-C₆ alkyl, C₂-C₆ alkenyl, C₂-C₆ alkynyl, halogen, C₁-C₆ haloalkyl, —CN, NO₂, —OR^{e}, —S(O)₂NR^{e}R^{f}, —C(O)R^{e}, —C(O)NR^{e}R^{f}, —NR^{e}R^{f} —N(R^{e})C(O)R^{f}, —(C₁-C₆ alkylenyl)-OR^{e}, —(C₁-C₆ alkylenyl)-C(O)NR^{e}R^{f}, a —(C₁-C₆alkylenyl)-NR^{e}R^{f}, and —(C₁-C₆ alkylenyl)-N(R^{e})C(O)R^{f};
R^{a} and R^{b}, at each occurrence, are independently selected from H, C₁-C₆ alkenyl, C₁-C₆ alkynyl, C₁-C₆ haloalkyl, R^{c}, and C₁-C₆ alkyl, wherein the C₁-C₆ alkyl is optionally substituted with one substituent selected from —OR^{e}, —NR^{e}R^{f}, —C(O)OR^{e}, —C(O)NR^{e}R^{f}, —S(O)₂R^{e}, —S(O)₂NR^{e}R^{f}, and R^{c};
R^{c} and R^{c′}, at each occurrence, are independently selected from aryl, heteroaryl, heterocycle, cycloalkyl, and cycloalkenyl, wherein each R^{c} group is optionally substituted with 1, 2, 3, 4, or 5 R^{d} groups;
R^{d}, at each occurrence, are independently selected from C₁-C₆ alkyl, C₂-C₆ alkenyl, C₂-C₆ alkynyl, halogen, C₁-C₆ haloalkyl, —CN, —NO₂, —OR^{e}, —S(O)₂NR^{e}R^{f}, —C(O)R^{e}, —C(O)NR^{e}R^{f}, —NR^{e}R^{f}, —N(R^{e})C(O)R^{f}, —(C₁-C₆ alkylenyl)-OR^{e}, —(C₁-C₆ alkylenyl)-C(O)NR^{e}R^{f}, —(C₁-C₆ alkylenyl)-NR^{e}R^{f}, and —(C₁-C₆ alkylenyl)-N(R^{e})C(O)R^{f}; and
R^{e} and R^{f}, at each occurrence, are independently selected from H, C₁-C₆ alkyl, C₁-C₆ cycloalkyl, aryl, heteroaryl and C₁-C₆ haloalkyl;
(b) an immune checkpoint inhibitor; and
(c) a pharmaceutically acceptable carrier.

28. (canceled)
